(19) United States
(12) Patent Application Publication
(10) Pub. No.: US 2024/0051955 A1
(43) Pub. Date: Feb. 15, 2024

(54) 4-AMINO-3-(4-PHENOXYPHENYL)-1,3-DIHYDRO-2H-IMIDAZO[4,5-C]PYRIDIN-2-ONE DERIVATIVES AND SALTS THEREOF

(71) Applicant: Genzyme Corporation, Cambridge, MA (US)

(72) Inventors: Catherine Arabeyre, Prades Le Lez (FR); Patricia Moliner, Perols (FR); Sabine Boisnard, Boulogne (FR); Damien Sallaberry, Le Plessis Robinson (FR); Serge Perard, Palaiseau (FR); Sebastien Roy, Sermaise (FR)

(73) Assignee: Genzyme Corporation, Cambridge, MA (US)

(21) Appl. No.: 18/268,577

(22) PCT Filed: Dec. 22, 2021
(86) PCT No.: PCT/US2021/064800

§ 371 Date: Jun. 20, 2023

Related U.S. Application Data
(60) Provisional application No. 63/245,288, filed on Sep. 17, 2021.
(60) Provisional application No. 63/130,010, filed on Dec. 23, 2020.

(51) Int. Cl. C07D 471/04

(52) U.S. Cl. CPC C07D 471/04

(57) ABSTRACT

The present disclosure relates to 4-amino-3-(4-phenoxyphenyl)-1,3-dihydro-2H-imidazo[4,5-c]pyridin-2-one variants or derivatives, and salts thereof, for use as agonists and antagonists. The disclosure further relates to compositions, methods of preparing, and methods of treatment.

This application claims priority of U.S. Provisional Application No. 63/130,010, filed Dec. 23, 2020, and U.S. Provisional Application No. 63/245,288, filed Sep. 17, 2021.

### FIELD

The present disclosure relates to 4-amino-3-(4-phenoxyphenyl)-1,3-dihydro-2H-imidazo[4,5-c]pyridin-2-one variants or derivatives, and salts thereof, for use as agonists and antagonists. The disclosure further relates to pharmaceutical compositions, methods of preparing, and methods of treatment.

### BACKGROUND

Small molecule drugs have found utility in a wide range of diseases and conditions. Some act as antagonists by blocking or dampening a biological response. Some act as agonists by activating a biological response. Others can function as both.

While many small molecule drugs are known, there is a need for new and different therapeutic agents. For example, according to National Center for Complementary and Integrative Health, about 2.5 million people world-wide, including some 400,000 people in the United States, suffer from multiple sclerosis (“MS”). While there are some drugs that are available for the treatment of MS, not everyone responds well to the available medications. Other examples of diseases or conditions where a need exists include autoimmune disorders such as lupus, pemphigus vulgaris, myasthenia gravis, Sjogren's syndrome, dry eye, multiple sclerosis, Wegener's granulomatosis, autoimmune hemolytic anemia, idiopathic thrombocytopenic purpura, Granulomatosis with Polyangiitis, or rheumatoid arthritis.

The present disclosure provides compounds that are tyrosine kinase inhibitors, in particular Bruton tyrosine kinase (“BTK”) inhibitors, and are therefore useful for the treatment of diseases such as cancer, autoimmune, inflammatory, and thromboembolic diseases. Also provided are pharmaceutical compositions containing such compounds and processes for preparing such compounds.

BTK, a member of the Tec family non-receptor tyrosine kinases, is essential for B cell signaling downstream from the B-cell receptor. It is expressed in B cells and other hematopoietic cells such as monocytes, macrophages and mast cells. It functions in various aspects of B cell function that maintain the B cell repertoire (see Gauld S. B. et al., B cell antigen receptor signaling: roles in cell development and disease. Science, 296:1641-2. 2002). B cells play a role in rheumatoid arthritis (see Perosa F., et al., CD20-depleting therapy in autoimmune diseases: from basic research to the clinic. J Intern Med. 267:260-77. 2010 and Dörner T, et al. Targeting B cells in immune-mediated inflammatory disease: a comprehensive review of mechanisms of action and identification of biomarkers. Pharmacol Ther. 125:464-75. 2010 and Honigberg, L., et. al., The selective BTK inhibitor PCI-32765 blocks B cell and mast cell activation and prevents mouse collagen-induced arthritis. Clin. Immunol 127 S1:S111. 2008) and in other autoimmune diseases such as systemic lupus erythematosus and cancers (see Shlomchik M. J., et. al., The role of B cells in lpr/lpr-induced autoimmunity. J. Exp Med. 180:1295-1306. 1994; Honigberg L. A., The Bruton tyrosine kinase inhibitor PCI-32765 blocks B-cell activation and is efficacious in models of autoimmune disease and B-cell malignancy. Proc. Natl. Acad. Sci. 107:13075-80. 2010; and Mina-Osorio P, et al., Suppression of glomerulonephritis in lupus-prone NZB×NZW mice by RN486, a selective inhibitor of Bruton's tyrosine kinase. Arthritis Rheum. 65: 2380-91. 2013).

There is also potential for BTK inhibitors for treating allergic diseases (see Honigberg, L., et. al, The selective BTK inhibitor PCI-32765 blocks B cell and mast cell activation and prevents mouse collagen induced arthritis. Clin. Immunol 127 S1:S111. 2008). It was noted that the irreversible inhibitor suppresses passive cutaneous anaphylaxis (PCA) induced by IgE antigen complex in mice. These findings are in agreement with those noted with BTK-mutant mast cells and knockout mice and suggest that BTK inhibitors may be useful for the treatment of asthma, an IgE-dependent allergic disease of the airway.

Accordingly, compounds that inhibit BTK would be useful in treatment for diseases such as autoimmune diseases, inflammatory diseases, and cancer.

It has been surprisingly discovered that certain 4-amino-3-(4-phenoxyphenyl)-1,3-dihydro-2H-imidazo[4,5-c]pyridin-2-one derivatives act as agonists, antagonists, or both on a number of receptors that are implicated in many of the diseases and disorders, including those listed above.

### SUMMARY

Described herein, in certain embodiments, are derivatives of 4-amino-3-(4-phenoxyphenyl)-1,3-dihydro-2H-imidazo[4,5-c]pyridin-2-one and salts thereof.

In one embodiment, disclosed herein is a compound of Formula I:

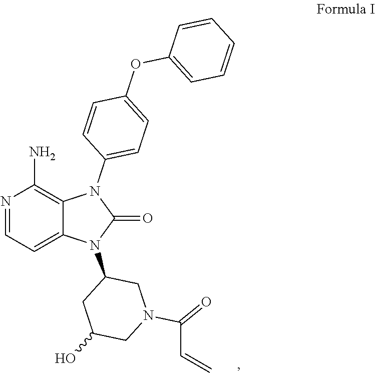 [SMILES: C=CC(=O)N1CC(O)C[C@@H](n2c(=O)n(-c3ccc(Oc4ccccc4)cc3)c3c(N)nccc32)C1]

or a salt thereof.

In one embodiment, disclosed herein is a compound of Formula Ia:

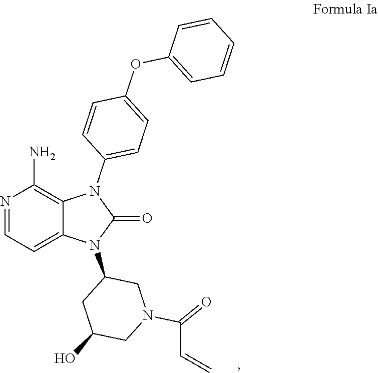 [SMILES: C=CC(=O)N1C[C@@H](O)C[C@@H](n2c(=O)n(-c3ccc(Oc4ccccc4)cc3)c3c(N)nccc32)C1]

or a salt thereof. In another embodiment, the compound of Formula Ia or salt thereof is about 90% pure.

In another embodiment, disclosed herein is a compound of Formula Ib:

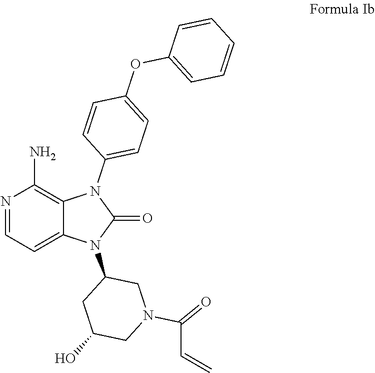 [SMILES: C=CC(=O)N1C[C@H](O)C[C@@H](n2c(=O)n(-c3ccc(Oc4ccccc4)cc3)c3c(N)nccc32)C1]

or a salt thereof, wherein the compound or salt thereof is about 90% pure.

In a further embodiment, disclosed herein is a compound of Formula II

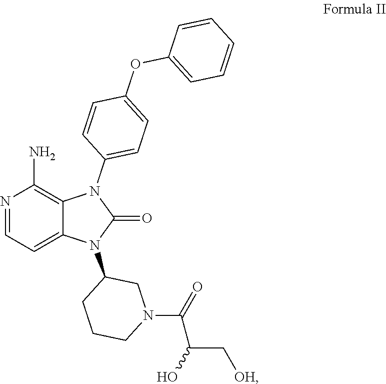 [SMILES: Nc1nccc2c1n(-c1ccc(Oc3ccccc3)cc1)c(=O)n2[C@@H]1CCCN(C(=O)C(O)CO)C1]

or salt thereof, wherein the compound or salt thereof is about 99.6% pure.

In another embodiment, disclosed herein is a composition comprising a pharmaceutically acceptable excipient and at least one compound chosen from:

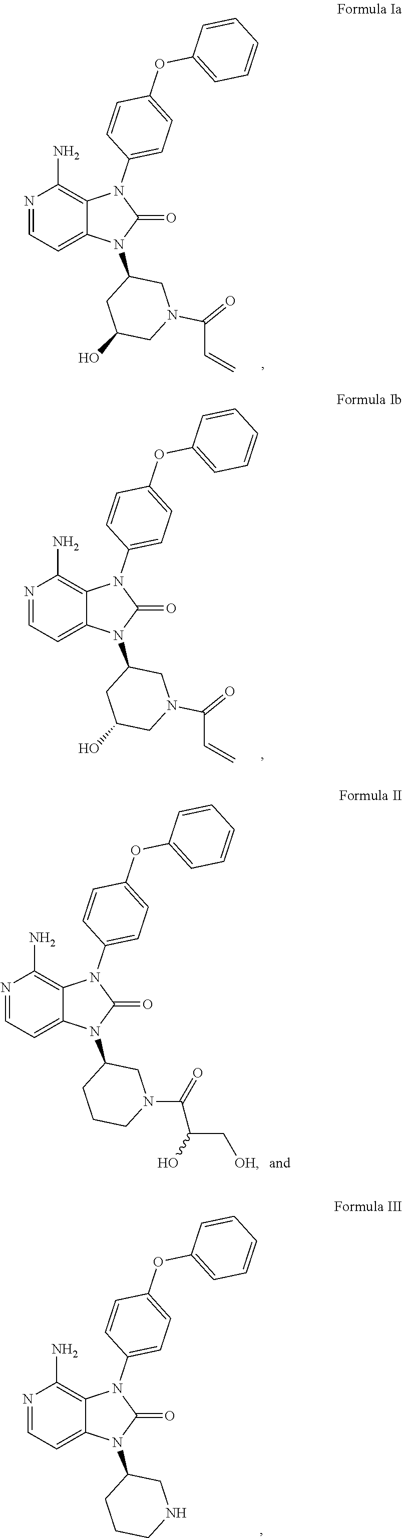

or a salt thereof.

In a further embodiment, disclosed herein is a method of preparing a compound of Formula Ia or salt thereof, the method comprising reacting a compound of Formula VIa

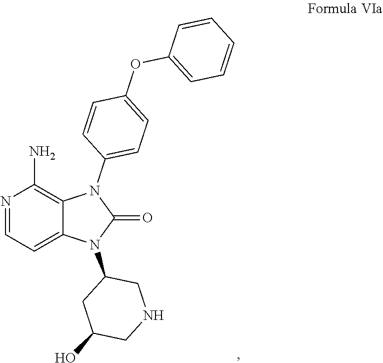 [SMILES: Nc1nccc2c1n(-c1ccc(Oc3ccccc3)cc1)c(=O)n2[C@H]1CNC[C@@H](O)C1]

with acryloyl chloride.

In a further embodiment, disclosed herein is a method of preparing a compound of Formula Ib or salt thereof, the method comprising reacting a compound of Formula VIb

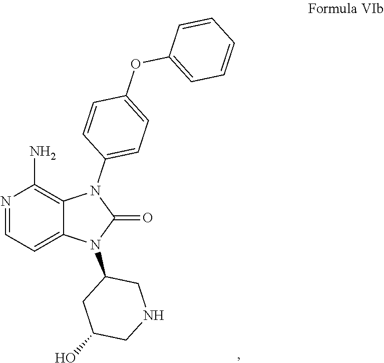 [SMILES: Nc1nccc2c1n(-c1ccc(Oc3ccccc3)cc1)c(=O)n2[C@H]1CNC[C@H](O)C1]

with acryloyl chloride.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing summary and the following detailed description are exemplary and explanatory only and are not restrictive of the claims.

### DESCRIPTION OF THE EMBODIMENTS

### I. Definitions

Unless otherwise stated, the following terms used in the specification and claims are defined for the purposes of this disclosure and have the following meaning:

A “pharmaceutically acceptable carrier” or “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier” or “a pharmaceutically acceptable excipient” as used in the specification and claims includes both one and more than one such carrier or excipient.

“Treating” or “treatment” of a disease includes:
(1) preventing the disease, e.g., causing the clinical symptoms of the disease not to develop in a patient, e.g., a human patient, that may have been exposed to or predisposed to the disease but does not yet experience or display symptoms of the disease;
(2) inhibiting the disease, e.g., arresting or reducing the development of the disease or its clinical symptoms; or
(3) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the 4-amino-3-(4-phenoxyphenyl)-1,3-dihydro-2H-imidazo[4,5-c]pyridin-2-one derivative or salt thereof, that, when administered to a patient, e.g., a human patient for treating a disease, is sufficient to affect such treatment for the disease. The “therapeutically effective amount” will vary depending on the compound, the disease and its severity and the age, weight, etc., of the patient, e.g., a human patient to be treated.

“about X % pure” or “about X % purity” (where “X” is a number) means that the referenced compound is present in about X % as determined by area via HPLC (detected by UV @254 nm) of the total peak area.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

“Or” is used in the inclusive sense, i.e., equivalent to “and/or,” unless the context requires otherwise.

### II. Compounds

In one aspect, provided herein are compounds that are 4-amino-3-(4-phenoxyphenyl)-1,3-dihydro-2H-imidazo[4,5-c]pyridin-2-one derivatives and salts thereof. In some embodiments, the compounds are about 50% pure. In some embodiments, the compounds are about 55% pure. In some embodiments, the compounds are about 60% pure. In some embodiments, the compounds are about 65% pure. In some embodiments, the compounds are about 70% pure. In some embodiments, the compounds are about 75% pure. In some embodiments, the compounds are about 80% pure. In some embodiments, the compounds are about 85% pure. In some embodiments, the compounds are about 90% pure. In some embodiments, the compounds are about 95% pure. In some embodiments, the compounds are about 98% pure. In some embodiments, the compounds are about 99% pure.

In one aspect, provided herein is a compound of Formula I:

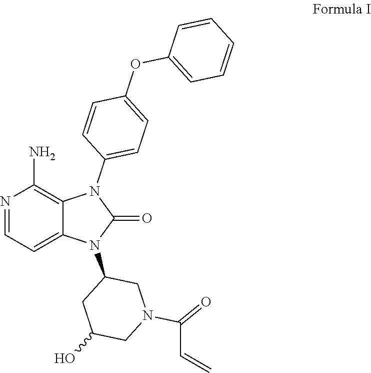 [SMILES: C=CC(=O)N1CC(O)C[C@@H](n2c(=O)n(-c3ccc(Oc4ccccc4)cc3)c3c(N)nccc32)C1]

or a salt thereof.

In one aspect, provided herein is a compound of Formula Ia:

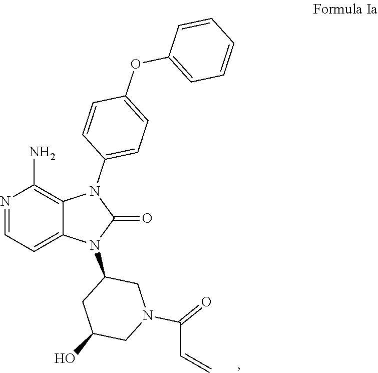 [SMILES: C=CC(=O)N1C[C@@H](O)C[C@@H](n2c(=O)n(-c3ccc(Oc4ccccc4)cc3)c3c(N)nccc32)C1]

or a salt thereof. In some embodiments, the compound of Formula Ia or salt thereof is about 90% pure. In some embodiments, the compound of Formula Ia or salt thereof is about 95% pure. In some embodiments, the compound of Formula Ia or salt thereof is about 98% pure. In some embodiments, the compound of Formula Ia or salt thereof is about 99% pure.

In one aspect, provided herein is a compound of Formula Ib:

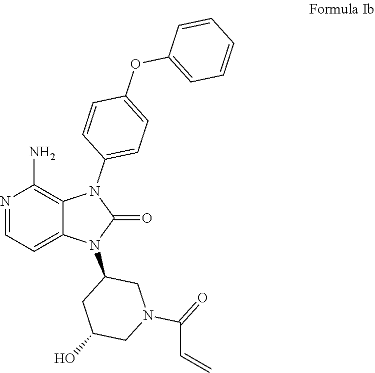 [SMILES: C=CC(=O)N1C[C@H](O)C[C@@H](n2c(=O)n(-c3ccc(Oc4ccccc4)cc3)c3c(N)nccc32)C1]

or a salt thereof, wherein the compound or salt thereof is about 90% pure. In some embodiments, the compound of Formula Ib or salt thereof is about 95% pure. In some embodiments, the compound of Formula Ib or salt thereof is about 98% pure. In some embodiments, the compound of Formula Ib or salt thereof is about 99% pure.

In one aspect, provided herein is a compound of Formula II

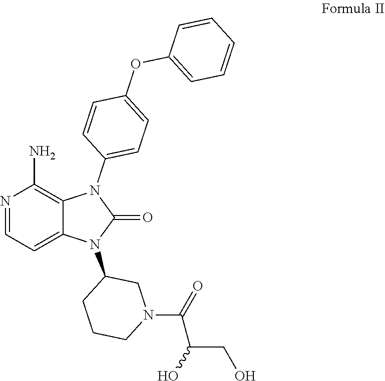 [SMILES: Nc1nccc2c1n(-c1ccc(Oc3ccccc3)cc1)c(=O)n2[C@@H]1CCCN(C(=O)C(O)CO)C1]

or salt thereof, wherein the compound or salt thereof is about 99.6% pure. In some embodiments, the compound is

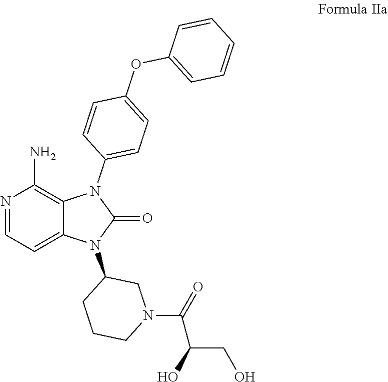 [SMILES: Nc1nccc2c1n(-c1ccc(Oc3ccccc3)cc1)c(=O)n2[C@@H]1CCCN(C(=O)[C@H](O)CO)C1]

or salt thereof, wherein the compound or salt thereof is about 90% pure. In some embodiments, the compound of Formula IIa or salt thereof is about 95% pure. In some embodiments, the compound of Formula IIa or salt thereof is about 98% pure. In some instances, the compound is

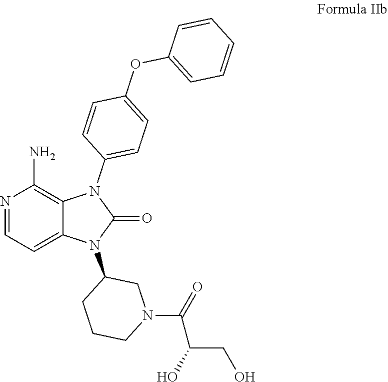 [SMILES: Nc1nccc2c1n(-c1ccc(Oc3ccccc3)cc1)c(=O)n2[C@@H]1CCCN(C(=O)[C@@H](O)CO)C1]

or salt thereof, wherein the compound or salt thereof is about 90% pure. In some embodiments, the compound of Formula IIb or salt thereof is about 95% pure.

In some embodiments, the compound of Formula IIb or salt thereof is about 98% pure.

In one aspect, the disclosure relates to a compound chosen from those of Formula III, Formula IV, and Formula V

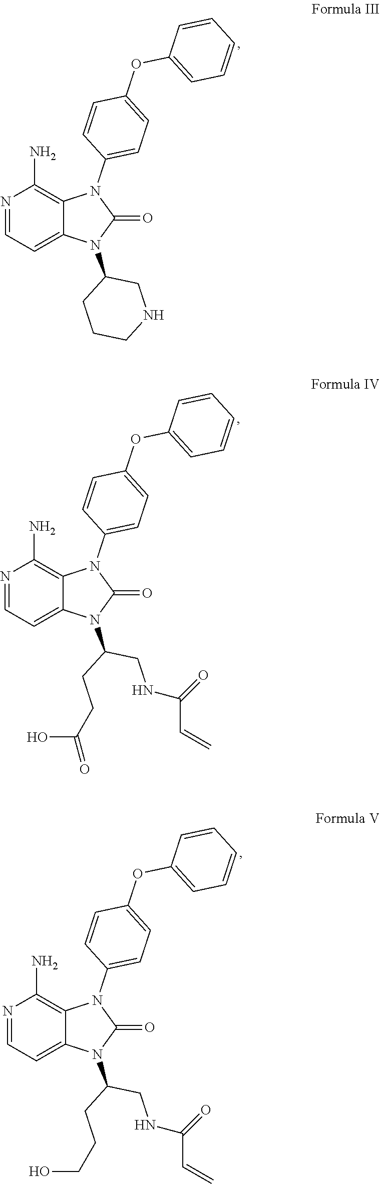 [SMILES: Nc1nccc2c1n(-c1ccc(Oc3ccccc3)cc1)c(=O)n2[C@@H]1CCCNC1 ; C=CC(=O)NC[C@@H](CCC(=O)O)n1c(=O)n(-c2ccc(Oc3ccccc3)cc2)c2c(N)nccc21 ; C=CC(=O)NC[C@@H](CCCO)n1c(=O)n(-c2ccc(Oc3ccccc3)cc2)c2c(N)nccc21]

or salts thereof.

### III. Compositions

In one aspect, provided herein are pharmaceutical compositions comprising a pharmaceutically acceptable excipient, and compounds which are variants or derivatives of 4-amino-3-(4-phenoxyphenyl)-1,3-dihydro-2H-imidazo[4,5-c]pyridin-2-one or salts thereof. These compounds were discovered by the present inventors as in vivo metabolites of (R)-1-(1-acryloylpiperidin-3-yl)-4-amino-3-(4-phenoxyphenyl)-1H-imidazo[4,5-c] pyridin-2(3H)-one.

In one embodiment, provided herein is a composition comprising a pharmaceutically acceptable excipient and at least one compound chosen from:

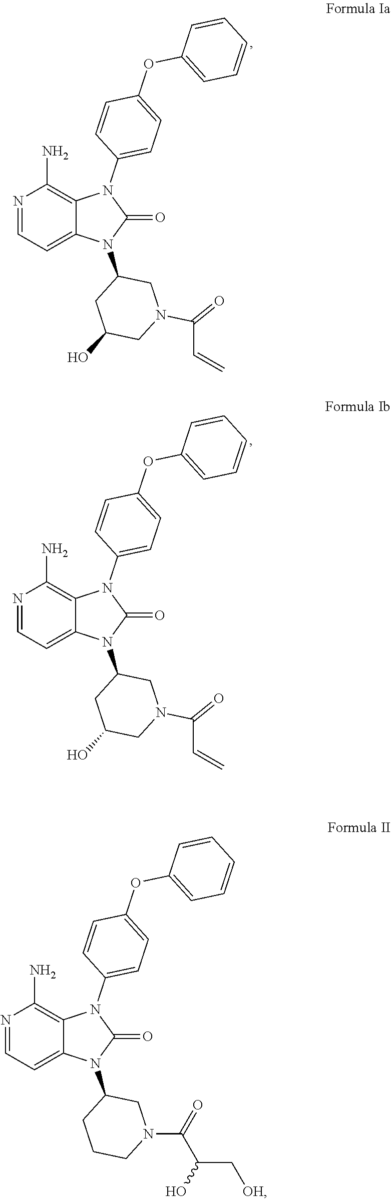

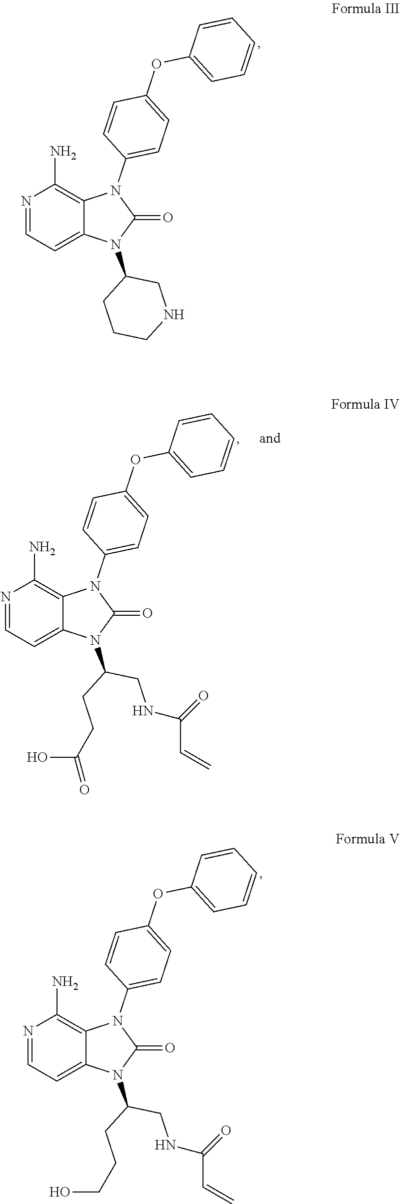 [SMILES: Nc1nccc2c1n(-c1ccc(Oc3ccccc3)cc1)c(=O)n2[C@@H]1CCCNC1 ; C=CC(=O)NC[C@@H](CCC(=O)O)n1c(=O)n(-c2ccc(Oc3ccccc3)cc2)c2c(N)nccc21 ; C=CC(=O)NC[C@@H](CCCO)n1c(=O)n(-c2ccc(Oc3ccccc3)cc2)c2c(N)nccc21]

or a salt thereof. In some embodiments, at least one compound or salt thereof is present in an effective amount.

In one embodiment, provided herein is a composition comprising a pharmaceutically acceptable excipient and at least one compound chosen from:

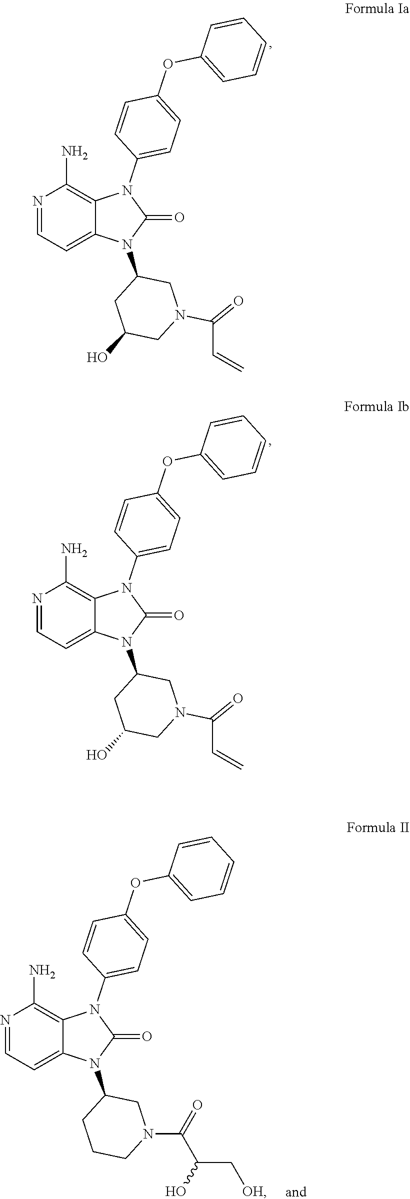

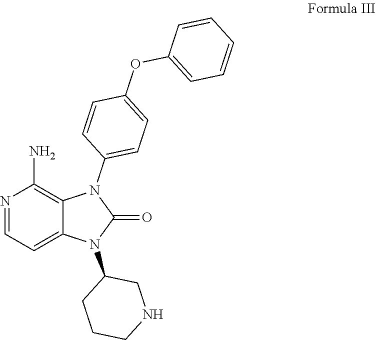 [SMILES: Nc1nccc2c1n(-c1ccc(Oc3ccccc3)cc1)c(=O)n2[C@@H]1CCCNC1]

or a salt thereof.

In some embodiments, at least one compound or salt thereof is present in an effective amount. In one embodiment, provided herein is a composition comprising a pharmaceutically acceptable excipient and a compound of Formula Ia or a salt thereof. In some embodiments, the compound of Formula Ia or salt thereof is at least about 98% pure.

In one embodiment, provided herein is a composition comprising a pharmaceutically acceptable excipient and a compound of Formula Ib or a salt thereof. In some embodiments, the compound of Formula Ib or salt thereof is at least about 99% pure.

Pharmaceutical compositions according to the disclosure may take a form suitable for oral, buccal, parenteral, nasal, topical, or rectal administration, or a form suitable for administration by inhalation.

The compounds described herein can be used in the preparation of a composition, such as a pharmaceutical composition, by combining the compound as an active ingredient with a pharmaceutically acceptable excipient. Some examples of materials which can serve as pharmaceutically acceptable excipients include: sugars, such as lactose, glucose and sucrose; starches, such as corn starch and potato starch; cellulose and its derivatives, such as sodium carboxymethyl cellulose, ethyl cellulose and cellulose acetate; surfactants, such as polysorbate 80 (i.e., Tween 80); powdered tragacanth; malt; gelatin; talc; suppository bases, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates or polyanhydrides; and other non-toxic compatible substances employed in pharmaceutical compositions. Pharmaceutical compositions may be prepared by known pharmaceutical methods. Suitable formulations can be found in, for example, Remington: The Science and Practice of Pharmacy, Lippincott Williams & Wilkins, 21st ed. (2005), which is incorporated herein by reference.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol and the like; and metal chelating agents, such as citric acid, ethylenediamine tetraacetic acid (EDTA), sorbitol, tartaric acid, phosphoric acid and the like.

Compositions of the disclosure suitable for oral administration may be in the form of capsules, cachets, pills, tablets, lozenges (using a flavored basis, usually sucrose and acacia or tragacanth), powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragées, powders, granules and the like), the active ingredient is mixed with one or more pharmaceutically-acceptable carriers, such as sodium citrate or dicalcium phosphate or any of the following: fillers or extenders, such as starches, lactose, sucrose, glucose, mannitol or silicic acid; binders, such as, for example, carboxymethylcellulose, alginates, gelatin, polyvinyl pyrrolidone, sucrose or acacia; humectants, such as glycerol; disintegrating agents, such as agar-agar, calcium carbonate, potato or tapioca starch, alginic acid, certain silicates and sodium carbonate; solution retarding agents, such as paraffin; absorption accelerators, such as quaternary ammonium compounds; wetting agents, such as, for example, cetyl alcohol, glycerol monostearate and non-ionic surfactants; absorbents, such as kaolin and bentonite clay; lubricants, such a talc, calcium stearate, magnesium stearate, solid polyethylene glycols, sodium lauryl sulfate and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets and other solid dosage forms of the pharmaceutical compositions of the present disclosure, such as dragées, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes or microspheres. They may be formulated for rapid release, e.g., freeze-dried. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

### IV. Methods of Treatment

In one aspect, provided herein are methods of treatment comprising administering to a patient, e.g., a human patient, in need thereof an effective amount of a derivative of 4-amino-3-(4-phenoxyphenyl)-1,3-dihydro-2H-imidazo[4,5-c]pyridin-2-one or salt thereof. In another aspect, provided herein are methods of treatment comprising administering to a patient, e.g., a human patient, in need thereof a composition comprising a pharmaceutically acceptable excipient and an effective amount of a derivative of 4-amino-3-(4-phenoxyphenyl)-1,3-dihydro-2H-imidazo[4,5-c]pyridin-2-one or salt thereof.

In some embodiments, the method is a method of treating MS. In some embodiments, the method is a method of treating autoimmune disorders such as rheumatoid arthritis and lupus.

In one aspect, provided herein is a method of treatment comprising administering to a patient, e.g., a human patient, in need thereof an effective amount of a compound of Formula Ia:

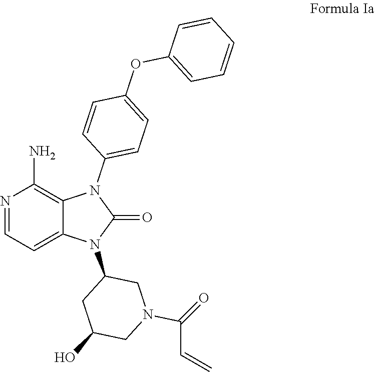 [SMILES: C=CC(=O)N1C[C@@H](O)C[C@@H](n2c(=O)n(-c3ccc(Oc4ccccc4)cc3)c3c(N)nccc32)C1]

or a salt thereof. In some embodiments, the compound of Formula Ia or salt thereof is about 90% pure. In some embodiments, the compound of Formula Ia or salt thereof is about 95% pure. In some embodiments, the compound of Formula Ia or salt thereof is about 98% pure. In some embodiments, the compound of Formula Ia or salt thereof is about 99% pure.

In some embodiments, the method of treatment comprising administering to a patient, e.g., a human patient, in need thereof an effective amount of a compound of Formula Ia or salt thereof is a method of treating MS. In some embodiments, the method of treatment comprising administering to a patient, e.g., a human patient, in need thereof an effective amount of a compound of Formula Ia or salt thereof is a method of treating autoimmune disorders such as rheumatoid arthritis and lupus.

In one aspect, provided herein is a method of treatment comprising administering to a patient, e.g., a human patient, in need thereof an effective amount of a compound of Formula Ib:

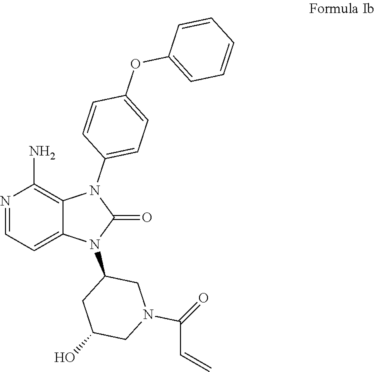 [SMILES: C=CC(=O)N1C[C@H](O)C[C@@H](n2c(=O)n(-c3ccc(Oc4ccccc4)cc3)c3c(N)nccc32)C1]

or a salt thereof, wherein the compound or salt thereof is about 90% pure. In some embodiments, the compound of Formula Ib or salt thereof is about 95% pure. In some embodiments, the compound of Formula Ib or salt thereof is about 98% pure. In some embodiments, the compound of Formula Ib or salt thereof is about 99% pure.

In some embodiments, the method of treatment comprising administering to a patient, e.g., a human patient, in need thereof an effective amount of a compound of Formula Ib or salt thereof is a method of treating MS. In some embodiments, the method of treatment comprising administering to a patient, e.g., a human patient, in need thereof an effective amount of a compound of Formula Ia or salt thereof is a of treating autoimmune disorders such as rheumatoid arthritis and lupus.

In one aspect, provided herein is a method of treatment comprising administering to a patient, e.g., a human patient, in need thereof an effective amount of a compound of Formula II:

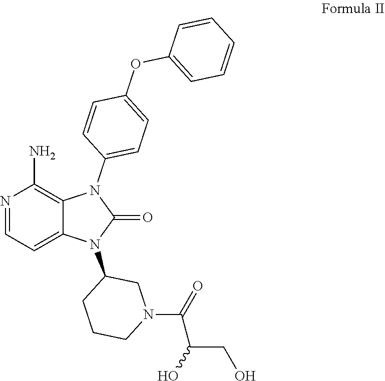 [SMILES: Nc1nccc2c1n(-c1ccc(Oc3ccccc3)cc1)c(=O)n2[C@@H]1CCCN(C(=O)C(O)CO)C1]

or salt thereof, wherein the compound or salt thereof is about 99.6% pure.

In some embodiments, the method of treatment comprising administering to a patient, e.g., a human patient, in need thereof an effective amount of a compound of Formula II or salt thereof is a method of treating MS. In some embodiments, the method of treatment comprising administering to a patient, e.g., a human patient, in need thereof an effective amount of a compound of Formula II or salt thereof is a of treating autoimmune disorders such as rheumatoid arthritis and lupus.

In some embodiments, the compound is

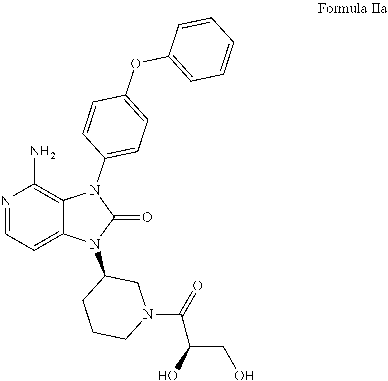 [SMILES: Nc1nccc2c1n(-c1ccc(Oc3ccccc3)cc1)c(=O)n2[C@@H]1CCCN(C(=O)[C@H](O)CO)C1]

or salt thereof, wherein the compound of Formula IIa or salt thereof is about 90% pure. In some embodiments, the compound of Formula IIa or salt thereof is about 95% pure. In some embodiments, the compound of Formula IIa or salt thereof is about 98% pure.

In some embodiments, the method of treatment comprising administering to a patient, e.g., a human patient, in need thereof an effective amount of a compound of Formula IIa or salt thereof is a method of treating MS. In some embodiments, the method of treatment comprising administering to a patient, e.g., a human patient, in need thereof an effective amount of a compound of Formula IIa or salt thereof is a of treating autoimmune disorders such as rheumatoid arthritis and lupus.

In some instances, the compound is

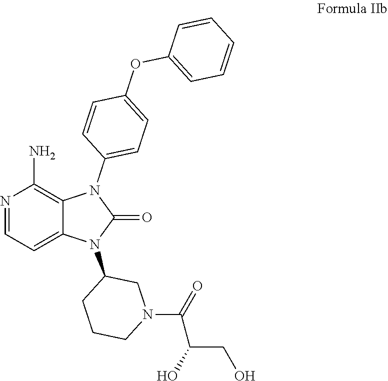 [SMILES: Nc1nccc2c1n(-c1ccc(Oc3ccccc3)cc1)c(=O)n2[C@@H]1CCCN(C(=O)[C@@H](O)CO)C1]

or salt thereof, wherein the compound of Formula IIb or salt thereof is about 90% pure. In some embodiments, the compound of Formula IIb or salt thereof is about 95% pure. In some embodiments, the compound of Formula IIb or salt thereof is about 98% pure.

In some embodiments, the method of treatment comprising administering to a patient, e.g., a human patient, in need thereof an effective amount of a compound of Formula IIb or salt thereof is a method of treating MS. In some embodiments, the method of treatment comprising administering to a patient, e.g., a human patient, in need thereof an effective amount of a compound of Formula IIb or salt thereof is a of treating an autoimmune disorder such as rheumatoid arthritis and lupus.

In one aspect, provided herein is a method of treatment comprising administering to a patient, e.g., a human patient, in need thereof a composition comprising a pharmaceutically acceptable excipient and an effective amount of at least one compound chosen from

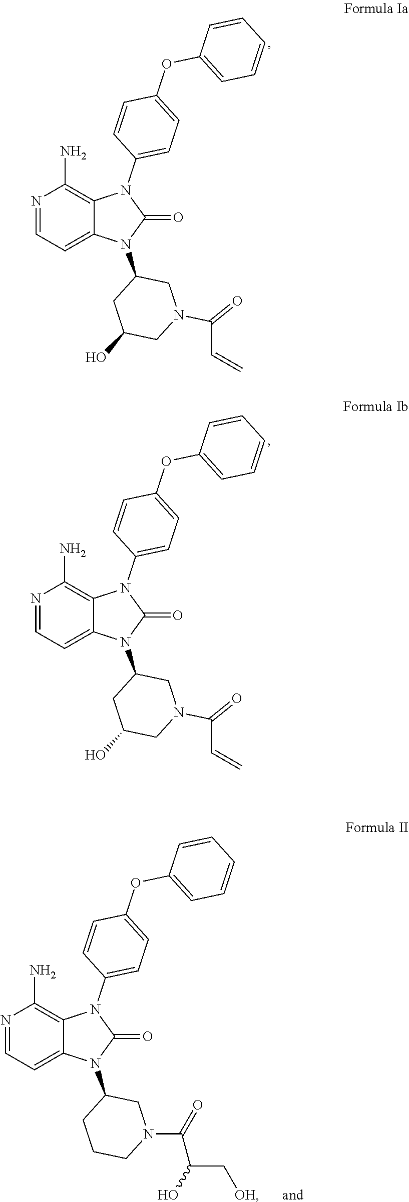

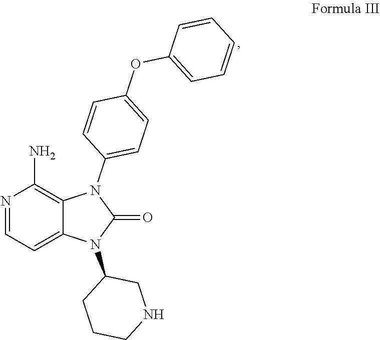 [SMILES: Nc1nccc2c1n(-c1ccc(Oc3ccccc3)cc1)c(=O)n2[C@@H]1CCCNC1]

or salt thereof. In some embodiments, at least one compound or salt thereof is present in an effective amount. In some embodiments, the compound or salt thereof is about 90% pure. In some embodiments, the compound or salt thereof is about 95% pure. In some embodiments, the compound or salt thereof is about 98% pure. In some embodiments, the compound or salt thereof is about 99% pure.

In some embodiments, the method of treatment comprising administering to a patient, e.g., a human patient, in need thereof a composition comprising a pharmaceutically acceptable excipient and an effective amount of at least one compound chosen from Formula Ia, Formula Ib, Formula II, and Formula III or salt thereof is a method of treating MS. In some embodiments, the method of treatment comprising administering to a patient, e.g., a human patient, in need thereof a composition comprising a pharmaceutically acceptable excipient and an effective amount of at least one compound chosen from Formula Ia, Formula Ib, Formula II, and Formula III, or salt thereof is a method of treating an autoimmune disorder such as rheumatoid arthritis and lupus.

In one aspect, provided herein are compositions for use as a medicament for treating a patient, e.g., a human patient, in need thereof comprising an effective amount of a derivative of 4-amino-3-(4-phenoxyphenyl)-1,3-dihydro-2H-imidazo[4,5-c]pyridin-2-one or salt thereof. In another aspect, provided herein are compositions for use as a medicament for treating a patient, e.g., a human patient, in need thereof comprising a pharmaceutically acceptable excipient and an effective amount of a derivative of 4-amino-3-(4-phenoxyphenyl)-1,3-dihydro-2H-imidazo[4,5-c]pyridin-2-one or salt thereof.

In some embodiments, the medicament is for treating MS. In some embodiments, the medicament is for treating autoimmune disorders such as rheumatoid arthritis and lupus.

In one aspect, provided herein is a composition for use as a medicament for treating a patient, e.g., a human patient, in need thereof comprising an effective amount of a compound of Formula Ia:

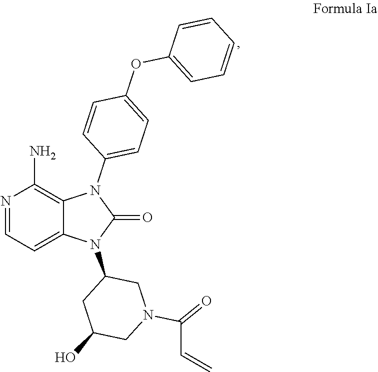 [SMILES: C=CC(=O)N1C[C@@H](O)C[C@@H](n2c(=O)n(-c3ccc(Oc4ccccc4)cc3)c3c(N)nccc32)C1]

or a salt thereof. In some embodiments, the compound of Formula Ia or salt thereof is about 90% pure. In some embodiments, the compound of Formula Ia or salt thereof is about 95% pure. In some embodiments, the compound of Formula Ia or salt thereof is about 98% pure. In some embodiments, the compound of Formula Ia or salt thereof is about 99% pure.

In some embodiments, the composition for use as a medicament for treating a patient, e.g., a human patient, in need thereof comprises an effective amount of a compound of Formula Ia or salt thereof for treating MS. In some embodiments, the composition for use as a medicament for treating a patient, e.g., a human patient, in need thereof comprises an effective amount of a compound of Formula Ia or salt thereof for treating autoimmune disorders such as rheumatoid arthritis and lupus.

In one aspect, provided herein is a composition for use as a medicament for treating a patient, e.g., a human patient, in need thereof comprising an effective amount of a compound of Formula Ib:

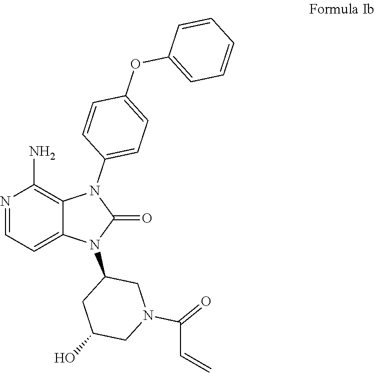 [SMILES: C=CC(=O)N1C[C@H](O)C[C@@H](n2c(=O)n(-c3ccc(Oc4ccccc4)cc3)c3c(N)nccc32)C1]

or a salt thereof, wherein the compound or salt thereof is about 90% pure. In some embodiments, the compound of Formula Ib or salt thereof is about 95% pure. In some embodiments, the compound of Formula Ib or salt thereof is about 98% pure. In some embodiments, the compound of Formula Ib or salt thereof is about 99% pure.

In some embodiments, the composition for use as a medicament for treating a patient, e.g., a human patient, in need thereof comprises an effective amount of a compound of Formula Ia or salt thereof for treating MS. In some embodiments, the composition for use as a medicament for treating a patient, e.g., a human patient, in need thereof comprises an effective amount of a compound of Formula Ia or salt thereof for treating autoimmune disorders such as rheumatoid arthritis and lupus.

In one aspect, provided herein is a composition for use as a medicament for treating a patient, e.g., a human patient, in need thereof comprising an effective amount of a compound of Formula II:

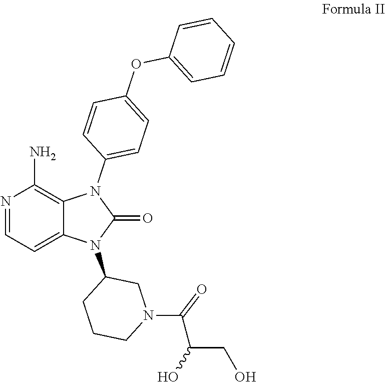 [SMILES: Nc1nccc2c1n(-c1ccc(Oc3ccccc3)cc1)c(=O)n2[C@@H]1CCCN(C(=O)C(O)CO)C1]

or salt thereof, wherein the compound or salt thereof is about 99.6% pure.

In some embodiments, the composition for use as a medicament for treating a patient, e.g., a human patient, in need thereof comprises an effective amount of a compound of Formula II or salt thereof for treating MS. In some embodiments, the composition for use as a medicament for treating a patient, e.g., a human patient, in need thereof comprises an effective amount of a compound of Formula II or salt thereof for treating autoimmune disorders such as rheumatoid arthritis and lupus.

In some embodiments, the compound is

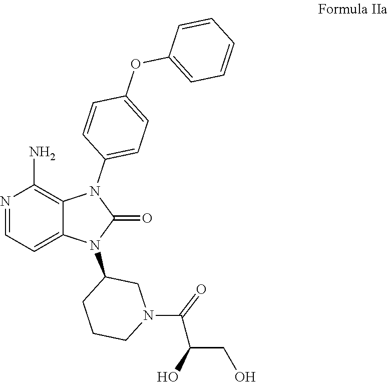 [SMILES: Nc1nccc2c1n(-c1ccc(Oc3ccccc3)cc1)c(=O)n2[C@@H]1CCCN(C(=O)[C@H](O)CO)C1]

or salt thereof, wherein the compound of Formula IIa or salt thereof is about 90% pure. In some embodiments, the compound of Formula IIa or salt thereof is about 95% pure. In some embodiments, the compound of Formula IIa or salt thereof is about 98% pure.

In some embodiments, the composition for use as a medicament for treating a patient, e.g., a human patient, in need thereof comprises an effective amount of a compound of Formula IIa or salt thereof for treating MS. In some embodiments, the composition for use as a medicament for treating a patient, e.g., a human patient, in need thereof comprises an effective amount of a compound of Formula IIa or salt thereof for treating autoimmune disorders such as rheumatoid arthritis and lupus.

In some instances, the compound is

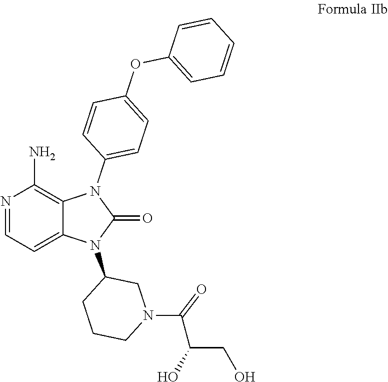 [SMILES: Nc1nccc2c1n(-c1ccc(Oc3ccccc3)cc1)c(=O)n2[C@@H]1CCCN(C(=O)[C@@H](O)CO)C1]

or salt thereof, wherein the compound of Formula IIb or salt thereof is about 90% pure. In some embodiments, the compound of Formula IIb or salt thereof is about 95% pure. In some embodiments, the compound of Formula IIb or salt thereof is about 98% pure.

In some embodiments, the composition for use as a medicament for treating a patient, e.g., a human patient, in need thereof comprises an effective amount of a compound of Formula IIb or salt thereof for treating MS. In some embodiments, the composition for use as a medicament for treating a patient, e.g., a human patient, in need thereof comprises an effective amount of a compound of Formula IIb or salt thereof for treating autoimmune disorders such as rheumatoid arthritis and lupus.

In one aspect, provided herein is a composition for use as a medicament for treating a patient, e.g., a human patient, in need thereof comprising a pharmaceutically acceptable excipient and an effective amount of at least one compound chosen from

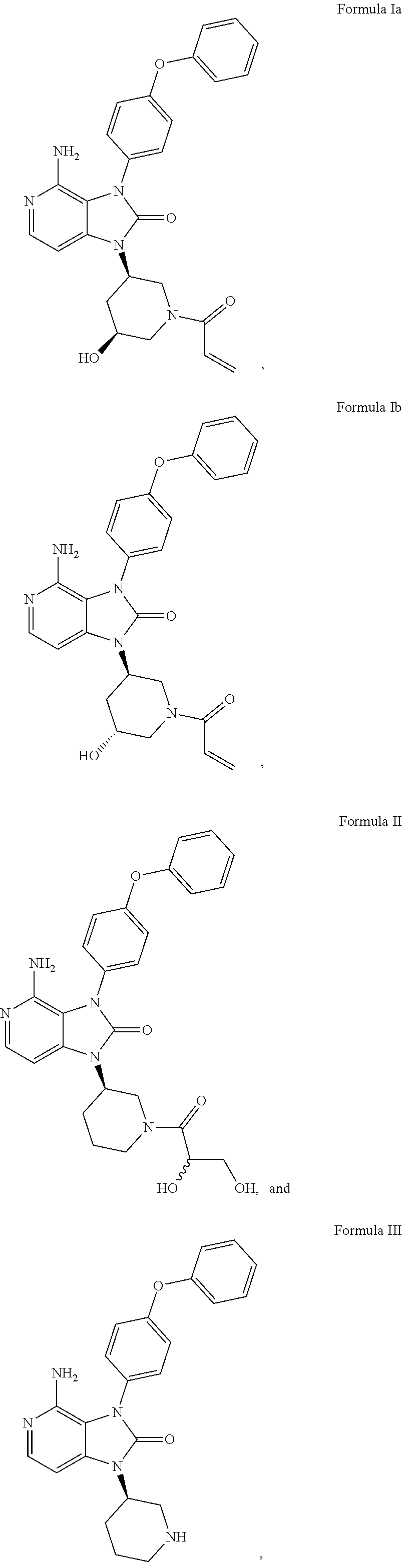

or salt thereof. In some embodiments, at least one compound or salt thereof is present in an effective amount. In some embodiments, the compound or salt thereof is about 90% pure. In some embodiments, the compound or salt thereof is about 95% pure. In some embodiments, the compound or salt thereof is about 98% pure. In some embodiments, the compound or salt thereof is about 99% pure.

In some embodiments, the composition for use as a medicament for treating a patient, e.g., a human patient, in need thereof comprises a pharmaceutically acceptable excipient and an effective amount of at least one compound chosen from Formula Ia, Formula Ib, Formula II, and Formula III or salt thereof is for treating MS. In some embodiments, the composition for use as a medicament for treating a patient, e.g., a human patient, in need thereof comprises a pharmaceutically acceptable excipient and an effective amount of at least one compound chosen from Formula Ia, Formula Ib, Formula II, and Formula III, and/or salt thereof is for treating an autoimmune disorder such as rheumatoid arthritis and lupus.

### V. Methods of Synthesis

In one aspect, provided herein are methods of synthesizing and preparing derivatives of 4-amino-3-(4-phenoxyphenyl)-1,3-dihydro-2H-imidazo[4,5-c]pyridin-2-one or salt thereof.

In one embodiment, provided herein is a method of preparing a compound of Formula Ia or salt thereof

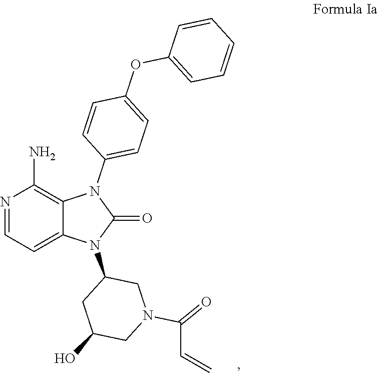 [SMILES: C=CC(=O)N1C[C@@H](O)C[C@@H](n2c(=O)n(-c3ccc(Oc4ccccc4)cc3)c3c(N)nccc32)C1]

the method comprising reacting a compound of Formula VIa

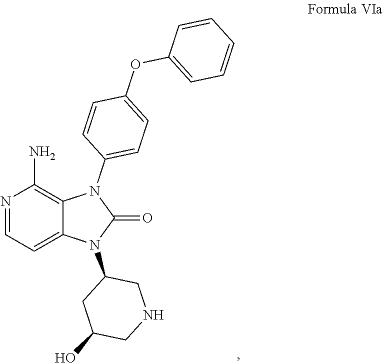 [SMILES: Nc1nccc2c1n(-c1ccc(Oc3ccccc3)cc1)c(=O)n2[C@H]1CNC[C@@H](O)C1]

with acryloyl chloride.

In some embodiments, the method further comprises preparing a compound of Formula VIa from a compound of Formula IX

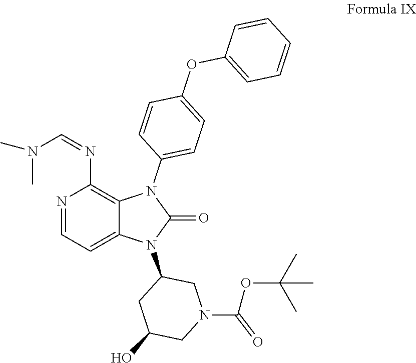 [SMILES: CN(C)/C=N\c1nccc2c1n(-c1ccc(Oc3ccccc3)cc1)c(=O)n2[C@@H]1C[C@H](O)CN(C(=O)OC(C)(C)C)C1]

by acid-based deprotection.

In one embodiment, provided herein is a method of preparing a compound of Formula Ib or salt thereof

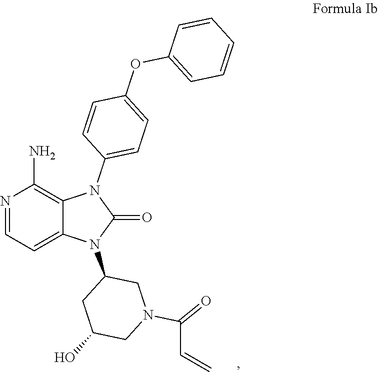 [SMILES: C=CC(=O)N1C[C@H](O)C[C@@H](n2c(=O)n(-c3ccc(Oc4ccccc4)cc3)c3c(N)nccc32)C1]

the method comprising reacting a compound of Formula VIb

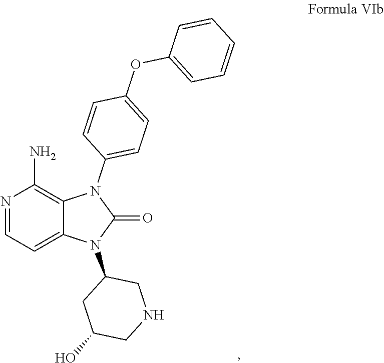 [SMILES: Nc1nccc2c1n(-c1ccc(Oc3ccccc3)cc1)c(=O)n2[C@H]1CNC[C@H](O)C1]

with acryloyl chloride.

In some embodiments, the method further comprises preparing a compound of Formula VIb from a compound of Formula VII

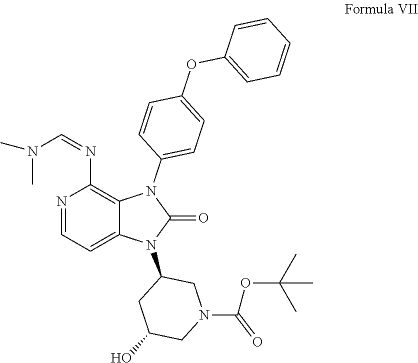 [SMILES: CN(C)/C=N\c1nccc2c1n(-c1ccc(Oc3ccccc3)cc1)c(=O)n2[C@@H]1C[C@@H](O)CN(C(=O)OC(C)(C)C)C1]

by acid-based deprotection.

In some embodiments, the method further comprises preparing a compound of Formula VII from a compound of Formula VIII

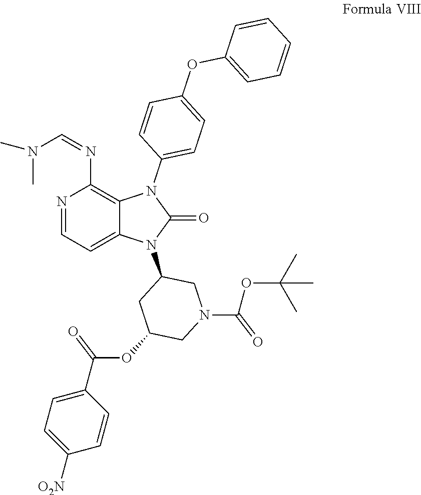 [SMILES: CN(C)/C=N\c1nccc2c1n(-c1ccc(Oc3ccccc3)cc1)c(=O)n2[C@@H]1C[C@@H](OC(=O)c2ccc([N+](=O)[O-])cc2)CN(C(=O)OC(C)(C)C)C1]

by deprotection using a base.

In some embodiments, the method further comprises preparing a compound of Formula VIII from a compound of Formula IX

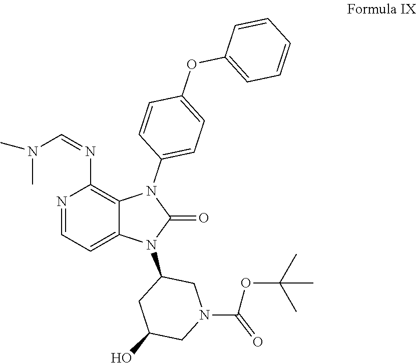 [SMILES: CN(C)/C=N\c1nccc2c1n(-c1ccc(Oc3ccccc3)cc1)c(=O)n2[C@@H]1C[C@H](O)CN(C(=O)OC(C)(C)C)C1]

with triphenylphosphine and an azodicarboxylate.

In some embodiments, the azodicarboxylate is chosen from diethyl azodicarboxylate and diisopropyl azodicarboxylate. In some embodiments, the azodicarboxylate is diisopropyl azodicarboxylate.

In one embodiment, provided herein is a method of preparing a compound of Formula IV or salt thereof

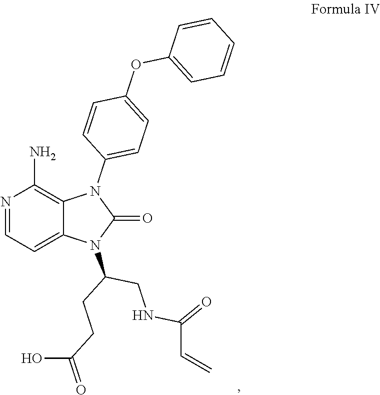 [SMILES: C=CC(=O)NC[C@@H](CCC(=O)O)n1c(=O)n(-c2ccc(Oc3ccccc3)cc2)c2c(N)nccc21]

the method comprising reacting (4R)-5-amino-4-[4-amino-2-oxo-3-(4-phenoxyphenyl)imidazo[4,5-c]pyridin-1-yl]pentanoic acid with acryloyl chloride.

In some embodiments, the method further comprises preparing (4R)-5-amino-4-[4-amino-2-oxo-3-(4-phenoxyphenyl)imidazo[4,5-c]pyridin-1-yl]pentanoic acid, or a salt thereof, from (4R)-4-[4-[bis(tert-butoxycarbonyl)amino]-2-oxo-3-(4-phenoxyphenyl)imidazo[4,5-c]pyridin-1-yl]-5-(tert-butoxycarbonylamino)pentanoic acid by acid deprotection.

In some embodiments, the method further comprises preparing from (4R)-4-[4-[bis(tert-butoxycarbonyl)amino]-2-oxo-3-(4-phenoxyphenyl)imidazo[4,5-c]pyridin-1-yl]-5-(tert-butoxycarbonylamino)pentanoic acid, or a salt thereof, from tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate by ring-opening in the presence of a base.

In some embodiments, the method further comprises preparing tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate, or salt thereof, from tert-butyl (3R)-3-[4-[bis(tert-butoxycarbonyl)amino]-2-oxo-3-(4-phenoxyphenyl)imidazo[4,5-c]pyridin-1-yl]piperidine-1-carboxylate by an electrochemical reaction utilizing a mediator and an electrolyte. In some embodiments, the mediator is quinucleidine. In further embodiments, the electrolyte is tetramethylammonium tetrafluoroborate.

In one embodiment, provided herein is a method of preparing a compound of Formula V or salt thereof

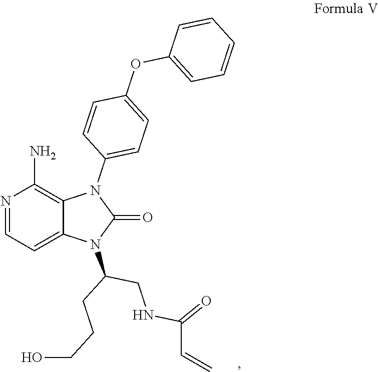 [SMILES: C=CC(=O)NC[C@@H](CCCO)n1c(=O)n(-c2ccc(Oc3ccccc3)cc2)c2c(N)nccc21]

the method comprising reacting 4-amino-1-[(1R)-1-(aminomethyl)-4-hydroxy-butyl]-3-(4-phenoxyphenyl)imidazo[4,5-c]pyridin-2-one with acryloyl chloride.

In some embodiments, the method further comprises preparing 4-amino-1-[(1R)-1-(aminomethyl)-4-hydroxy-butyl]-3-(4-phenoxyphenyl)imidazo[4,5-c]pyridin-2-one, or salt thereof, from tert-butyl N-tert-butoxycarbonyl-N-[1-[(1R)-1-[(tert-butoxycarbonylamino)methyl]-4-hydroxy-butyl]-2-oxo-3-(4-phenoxyphenyl)imidazo[4,5-c]pyridin-4-yl]carbamate by acid deprotection.

In some embodiment, the method further comprises preparing tert-butyl N-tert-butoxycarbonyl-N-[1-[(1R)-1-[(tert-butoxycarbonylamino)methyl]-4-hydroxy-butyl]-2-oxo-3-(4-phenoxyphenyl)imidazo[4,5-c]pyridin-4-yl]carbamate, or salt thereof, from tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate by ring-opening in the presence of a base.

In some embodiment, the method further comprises preparing tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate, or salt thereof, from tert-butyl (3R)-3-[4-[bis(tert-butoxycarbonyl)amino]-2-oxo-3-(4-phenoxyphenyl)imidazo[4,5-c]pyridin-1-yl]piperidine-1-carboxylate by an electrochemical reaction utilizing a mediator and an electrolyte. In some embodiments, the mediator is quinucleidine. In further embodiments, the electrolyte is tetramethylammonium tetrafluoroborate.

TABLE 1
Abbreviations and names used in the following examples and
elsewhere herein include:

| Abbreviation | Definition |
|---|---|
| AcOEt and EtOAc | Ethyl acetate |
| ATP | Adenosine triphosphate |
| Boc | t-Butyloxycarbonyl protecting group |
| BTK | Bruton's tyrosine kinase |
| DCM | Dichloromethane |
| DIAD | Diisopropyl azodicarboxylate |
| DIEA | N,N-Diisopropylethylamine |
| DIPEA | N,N-diisopropylethylamine |
| DMAP | 4-Dimethylaminopyridine |
| DMF | N,N-Dimethylformamide |
| DMF DMA | N,N-dimethylformamide dimethyl acetal |
| DMSO | Dimethyl sulfoxide |
| HFIP | 1,1,1,3,3,3-hexafluoropropan-2-ol |
| HPLC | High-performance liquid chromatography |
| HOBt | Hydroxybenzotriazole |
| MeOH | Methanol |
| MS | Molecular sieve |
| MTBE | Methyl t-butyl ether |
| PMB | p-Methoxybenzyl protecting group |
| SGC | Silica gel chromatography |
| tBuOH | t-Butyl alcohol |
| TFA | Trifluoroacetic acid |
| THF | Tetrahydrofuran |
| TLC | Thin layer chromatography |

### EXAMPLES

### Example 1. Synthesis of 4-amino-1-[(3R,5S)-5-hydroxy-1-prop-2-enoyl-3-piperidyl]-3-(4-phenoxyphenyl)imidazo[4,5-c]pyridin-2-one and 4-amino-1-[(3R,5R)-5-hydroxy-1-prop-2-enoyl-3-piperidyl]-3-(4-phenoxyphenyl)imidazo[4,5-c]pyridin-2-one

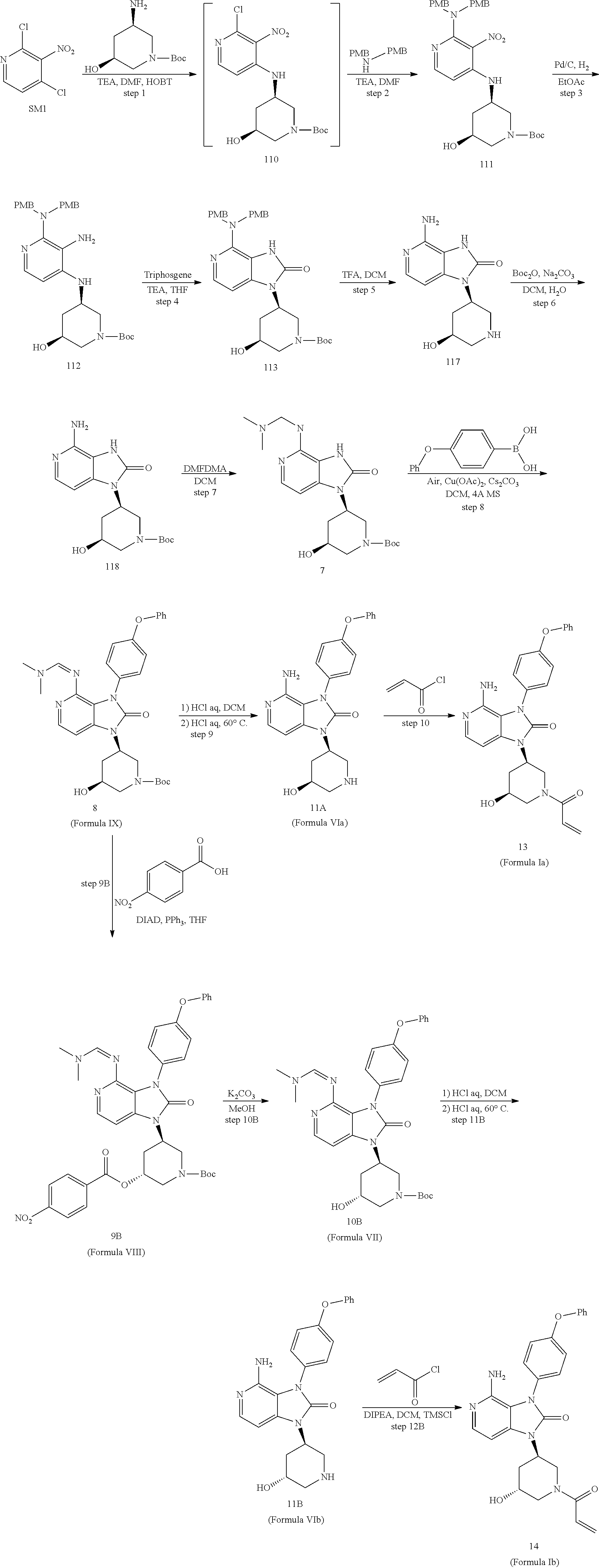

Compounds of Formula Ia and Formula Ib were synthesized as shown in the Scheme above (referred to as compounds 13 and 14 respectively) and as described in more detail below.

Step 1: A reactor under nitrogen atmosphere was charged with DMF (21.5 mL). 2,4-dichloro 3 nitropyridine (SM1) (4.3 g, 22.28 mmol, 1.0 eq) was added at 20±5° C. After stirring for 5 minutes, triethylamine (4.75 mL, 33.81 mmol, 1.5 eq) was added and the mixture was cooled to 0±5° C. After adding HOBt (301 mg, 2.23 mmol, 0.1 eq), a solution of tert-butyl (3R,5S)-3-amino-5-hydroxy-piperidine-1-carboxylate (4.8 g, 22.28 mmol, 1.0 eq) in DMF (64.5 mL) was added dropwise. During addition of the reagents, the temperature was maintained between −10° C. and 0° C. The reaction mixture was stirred for 2 hours at 0±5° C., then for 4 hours at 20±5° C. HPLC analysis was performed to check for formation of tert-butyl (3R,5S)-3-[[2-(benzotriazol-1-yloxy)-3-nitro-4-pyridyl]amino]-5-hydroxy-piperidine-1-carboxylate (110).

Step 2: Next step was immediately carried out in one pot by charging triethylamine (4.75 mL, 33.81 mmol, 1.5 eq) and 1-(4-methoxyphenyl)-N-[(4-methoxyphenyl)methyl]methanamine (5.7 g, 22.28 mmol, 1.0 eq). The reaction solution was warmed to 40˜45° C. and stirred at this temperature for 62 hours. HPLC analysis was performed to check that below 1% of intermediate was left. After cooling to 10˜20° C., EtOAc (129 mL) and water (129 mL) were added to the solution, which was stirred for 30 minutes at 10˜20° C. The organic phase was separated and washed with CaCl₂ aq. (5%, 43 mL×5). The organic phase was concentrated under vacuum below 35-C to afford tert-butyl (3R,5S)-3-[[2-[bis[(4-methoxyphenyl)methyl]amino]-3-nitro-4-pyridyl]amino]-5-hydroxy-piperidine-1-carboxylate (111) in EtOAc solution (brown solution) (94.3% purity), which was directly used in next step.

Step 3: Tert-butyl (3R,5S)-3-[[2-[bis[(4-methoxyphenyl)methyl]amino]-3-nitro-4-pyridyl]amino]-5-hydroxy-piperidine-1-carboxylate (111) (14.7 g, 24.76 mmol, 1 eq) (theoretical quantity) was dissolved in AcOEt (73.5 mL) and charged in a reactor. The reactor was inerted under vacuum and filled with nitrogen for five times, wet 10% Pd/C (2.64 g, 20 wt. %) was added, the reactor was inerted under vacuum and filled with nitrogen for five times and the reactor was then inerted under vacuum and filled with hydrogen for three times. The reaction mass was maintained under hydrogen pressure at 20±5° C. and stirred for 22 hours. HPLC analysis was performed to check the disappearance of starting material. The suspension was filtered, the cake was washed with EtOAc (73.5 mL×5). The filtrate was concentrated under vacuum to afford tert-butyl (3R,5S)-3-[[3-amino-2-[bis[(4-methoxyphenyl)methyl]amino]-4-pyridyl]amino]-5-hydroxy-piperidine-1-carboxylate (112) as a yellow foam solid (12 g, Yield=87%, 91.1% purity). This compound was used in next step without any further purification.

Step 4: In a reactor were charged THF (60 mL), tert-butyl (3R,5S)-3-[[2-[bis[(4-methoxyphenyl)methyl]amino]-3-nitro-4-pyridyl]amino]-5-hydroxy-piperidine-1-carboxylate (112) (12.0 g, 20.21 mmol, 1.0 eq) and triethylamine (4.4 mL, 40.42 mmol, 2.0 eq). The solution was stirred 10 min at 20±5° C., then triphosgene (1.07 mL, 6.46 mmol, 0.32 eq) was added in 3 batches and the resulting solution was stirred for 1.5 hour at 20˜25° C. HPLC analysis was performed to check that below 1% of intermediate was left. The solution was concentrated at 24 mL, CH₂Cl₂ (120 mL) and NaHCO₃ aq. (60 mL) were added, the mixture was stirred for 10 minutes. The organic phase was separated and concentrated under vacuum below 40° C. to provide the crude product, which was purified by SGC (CH₂Cl₂/MeOH=30/1) to afford tert-butyl (3R,5S)-3-[4-[bis[(4-methoxyphenyl)methyl] amino]-2-oxo-3H-imidazo[4,5-c]pyridin-1-yl]-5-hydroxy-piperidine-1-carboxylate (113) as a foam solid (10 g, yield=74%, 97.1% purity).

Step 5: A solution of tert-butyl (3R,5S)-3-[4-[bis[(4-methoxyphenyl)methyl]amino]-2-oxo-3H-imidazo[4,5-c]pyridin-1-yl]-5-hydroxy-piperidine-1-carboxylate (113) (10.0 g, 16.95 mmol, 1.0 eq) in CH₂Cl₂ (70 mL) was stirred 10 minutes at 20±5° C., then cooled to 5±5° C.; TFA (10 mL) was added slowly so the temperature was kept ≤10° C. The reaction solution was stirred for 30 minutes at 5±5° C., warmed to 40±5° C., then stirred for 14 hours. HPLC analysis was performed to check that starting material was ≤1.0%. The reaction mass was cooled to 15˜25° C. The pH was adjusted to 9-10 with NaCO₃ aq. The aqueous phase containing 4-amino-1-[(3R,5S)-5-hydroxy-3-piperidyl]-3H-imidazo[4,5-c]pyridin-2-one (117) was separated and kept to be engaged directly in next step.

Step 6: To the aqueous solution containing 4-amino-1-[(3R,5S)-5-hydroxy-3-piperidyl]-3H-imidazo[4,5-c]pyridin-2-one (117) cooled to 5±5° C. was added CH₂Cl₂ (40 mL), and a solution of Boc₂O (3.7 g, 16.95 mmol, 1.0 eq) in CH₂Cl₂ (10 mL) dropwise at 5±5° C. The solution was warmed to 15˜25° C. and stirred for 42 hours at 20˜25° C. HPLC analysis was performed to check that starting material was ≤3.0%. The aqueous phase was separated, extracted with CH₂Cl₂ (100 mL×3) and THF (100 mL×2). The organic phases were combined, concentrated under vacuum below 35-C to afford 5.4 g of tert-butyl (3R,5S)-3-(4-amino-2-oxo-3H-imidazo[4,5-c]pyridin-1-yl)-5-hydroxy-piperidine-1-carboxylate (118) as a solid with 92.2% purity (contains CH₂Cl₂/THF).

Step 7: In a reactor were charged CH₂Cl₂ (21.6 mL) and tert-butyl (3R,5S)-3-(4-amino-2-oxo-3H-imidazo[4,5-c]pyridin-1-yl)-5-hydroxy-piperidine-1-carboxylate (118) (5.4 g, 15.45 mmol, 1.0 eq). The suspension was stirred for 10 minutes. After adding N,N-dimethylformamide dimethyl acetal (6.1 mL, 46.35 mmol, 3.0 eq) at 20±5° C., the solution became clear. Upon stirring for 20 hours at 20±5° C., a solid precipitated. HPLC analysis was performed to check that starting material was ≤2.0%. MTBE (54 mL) was added and the reaction mass was stirred for 2 hours at 20±5° C. The suspension was filtered. The cake was washed with MTBE (21.6 mL), dried under vacuum at 35±5° C. for 8 hours to afford tert-butyl (3R,5S)-3-[4-[(Z)-dimethylaminomethyleneamino]-2-oxo-3H-imidazo[4,5-c]pyridin-1-yl]-5-hydroxy-piperidine-1-carboxylate (7) as an off-white solid (4.3 g, Yield=83%, 93.4% purity).

Step 8: In a reactor at 20±5° C. were charged CH₂Cl₂ (75 mL), Cu(OAc)₂ (561.2 mg, 3.09 mmol, 0.5 eq), 2,2′-Dipyridyl (482 mg, 3.09 mmol, 0.5 eq), Cs₂CO₃ (4.02 g, 12.36 mmol, 2.0 eq.), 4A MS (2.5 g, 1.0 w/w) and tert-butyl (3R,5S)-3-[4-[(Z)-dimethylaminomethyleneamino]-2-oxo-3H-imidazo[4,5-c]pyridin-1-yl]-5-hydroxy-piperidine-1-carboxylate (7) (2.5 g, 6.18 mmol, 1.0 eq). Compressed air (21% Oxygen and 79% Nitrogen) was bubbled to the reaction mass for 30 minutes at 20±5° C. To the reaction mixture, 4-Phenoxyphenylboronic (1.98 g, 9.27 mmol, 1.5 eq.) was added in four batches over 1.5 hours at 15˜25° C. Compressed air (21% Oxygen and 79% Nitrogen) was bubbled to the reaction mass for 5 hours at 20±5° C. HPLC analysis was performed to check that starting material was ≤5.0%. The suspension was filtered, the cake was washed with CH₂Cl₂ (25 mL×2). The filtrate was concentrated and the crude product was purified SGC (CH₂Cl₂/MeOH=50/1) to afford tert-butyl (3R,5S)-3-[4-[(Z)-dimethylaminomethyleneamino]-2-oxo-3-(4-phenoxyphenyl)imidazo[4,5-c]pyridin-1-yl]-5-hydroxy-piperidine-1-carboxylate (Compound of Formula IX, also referred to as (8) above) as solid with 97.5% purity and 47.2% yield.

Step 9: Tert-butyl (3R,5S)-3-[4-[(Z)-dimethylaminomethyleneamino]-2-oxo-3-(4-phenoxyphenyl)imidazo[4,5-c]pyridin-1-yl]-5-hydroxy-piperidine-1-carboxylate (8) (0.3 g, 0.52 mmol, 1.0 eq.) was dissolved in CH₂Cl₂ (1.5 mL). To this solution, aqueous HCl solution (3 N, 3 mL) was added dropwise at 20±5° C. The solution was stirred at 30˜35° C. for 2 hours and at 60˜65° C. for 14 hours. HPLC analysis was performed to check that starting material was ≤1.0%. To the solution cooled to 20˜25° C., CH₂Cl₂ (15 mL) and water (45 mL) were added. The solution was stirred for 10 minutes, then the aqueous phase was separated, extracted with CH₂Cl₂ (30 mL), and the pH of the aqueous phase was adjusted to 9-10 with Na₂CO₃ aq. The aqueous phase became pink. After stirring for 30 minutes, the organic phase was extracted and washed with brine (15 mL), dried over Na₂SO₄, filtered to provide 4-amino-1-[(3R,5S)-5-hydroxy-3-piperidyl]-3-(4-phenoxyphenyl)imidazo[4,5-c]pyridin-2-one (Compound of Formula VIa, also referred to as (11A) above) (81% purity) in solution in CH₂Cl₂. It was engaged directly in the Step 10.

Step 10: The previous solution of 4-amino-1-[(3R,5S)-5-hydroxy-3-piperidyl]-3-(4-phenoxyphenyl)imidazo[4,5-c]pyridin-2-one (11A) (164 mg, 0.39 mmol, 1 eq) in CH₂Cl₂ was cooled to 0±5° C., N,N-diisopropylethylamine (133 μL, 0.78 mmol, 2.0 eq) and trimethyl silyl chloride (50 μL, 0.39 mmol, 1.0 eq) in CH₂Cl₂ (2.5 ml) were charged, the solution was stirred for 30 minutes at 0±5° C. After cooling the solution to −20±5° C., acryloyl chloride (32 μL, 0.39 mmol, 1.0 eq) in CH₂Cl₂ (5 ml) was added dropwise. The reaction was quenched with water (20 mL), and the organic phase was separated and concentrated under vacuum below 35° C. to get the crude product. The crude product was purified by Prep-TLC (CH₂Cl₂/EtOH=15/1), the fraction was concentrated and CH₂Cl₂ was switched with EtOH. The solution was concentrated to 10 mL and n-heptane (15 ml) was charged. The solution was then concentrated to 10 mL and a solid precipitated. The precipitate was filtered to afford 80 mg of 4-amino-1-[(3R,5S)-5-hydroxy-1-prop-2-enoyl-3-piperidyl]-3-(4-phenoxyphenyl)imidazo[4,5-c]pyridin-2-one (Compound of Formula Ia, also referred to as (13) above) as an off-white solid with 98.1% purity and 43% yield. 1H NMR spectrum (500 MHz, DMSO D6, δ in ppm): 7.77 (d, 1H); 7.51-7.41 (m, 4H); 7.22 (t, 1H); 7.18-7.11 (m, 4H); 6.96 (br. m, 1H); 6.81 (m, 1H); 6.15 (d, 1H); 5.71 (dd, 1H); 5.28 (br. s, 1H); 4.83 (br. m, 21-1); 4.58 (br. d, 0.5H); 4.47 (br. d, 0.5H); 4.25 (br. qd, 1H); 4.15 (br. d, 0.5H); 4.1 (br. d, 0.5H); 3.69 (br. t, 0.5H); 3.60 (m, 1H); 3.27 (br. t, 0.5H); 2.95 (br. t, 0.5H); 2.49 (br. t, 0.5H); 2.28 (m, 1H); 2.13 (br. m, 1H)

Step 9B: In a reactor inerted with azote, THF (12.5 mL), DIAD (683 μL, 3.48 mmol, 4.0 eq), and Ph₃P (912 mg, 3.48 mmol, 4.0 eq) were charged. The reaction mass was cooled to 0˜5° C., then tert-butyl (3R,5S)-3-[4-[(Z)-dimethylaminomethyleneamino]-2-oxo-3-(4-phenoxyphenyl)imidazo[4,5-c]pyridin-1-yl]-5-hydroxy-piperidine-1-carboxylate (8) (0.5 g, 0.87 mmol, 1.0 eq) was added to the reactor. The reaction mass was stirred for 10 minutes at 0˜5° C., 4-nitro benzoic acid (291 mg, 1.74 mmol, 2.0 eq) was added to the reactor and the reaction was stirred a further 2 hours at 0˜5° C. The reaction mixture was then stirred 14 hours at 15˜20° C., concentrated under vacuum below 35° C. The residual was purified with SGC (CH₂Cl₂/MeOH=50/1) to afford crude tert-butyl (3R,5R)-3-[4-[(Z)-dimethylaminomethyleneamino]-2-oxo-3-(4-phenoxyphenyl)imidazo[4,5-c]pyridin-1-yl]-5-(4-nitrobenzoyl)oxy-piperidine-1-carboxylate (Compound of Formula VIII, also referred to as (9B) above) as a foam solid (purity 87%) which contains Ph₃PO. The Ph₃PO was hard to separate and this intermediate was not further purified.

Step 10B: MeOH (9 mL), tert-butyl (3R,5R)-3-[4-[(Z)-dimethylaminomethyleneamino]-2-oxo-3-(4-phenoxyphenyl)imidazo[4,5-c]pyridin-1-yl]-5-(4-nitrobenzoyl)oxy-piperidine-1-carboxylate (9B) (0.45 g, 0.62 mmol, 1.0 eq) and K₂CO₃ (258 mg, 1.87 mmol, 3.0 eq) were charged to a flask. The reaction mixture was stirred for 14 hours at 15˜20° C. HPLC analysis showed that no starting material was left. The suspension was filtered, the cake was washed with MeOH (2.25 mL), the filtrate was concentrated under vacuum below 35° C. to provide the crude product. The crude product was purified by SGC (CH₂Cl₂/MeOH=50/1) to afford tert-butyl (3R,5R)-3-[4-[(Z)-dimethylaminomethyleneamino]-2-oxo-3-(4-phenoxyphenyl)imidazo[4,5-c]pyridin-1-yl]-5-hydroxy-piperidine-1-carboxylate (Compound of Formula VII, also referred to as (10B) above) (0.16 g, yield 44%, 98.2% purity) as a powder solid.

Step 11B: To a flask were charged CH₂Cl₂ (1 mL), tert-butyl (3R,5R)-3-[4-[(Z)-dimethylaminomethyleneamino]-2-oxo-3-(4-phenoxyphenyl)imidazo[4,5-c]pyridin-1-yl]-5-hydroxy-piperidine-1-carboxylate (10B) (0.16 g, 0.28 mmol, 1 eq) and HCl aq (3.0M, 2 mL). The solution was stirred for 2 hours at 25˜30° C., then for 14 hours at 60˜65° C. HPLC analysis showed that no starting material remained. The reaction mixture was cooled to 15˜20° C., and CH₂Cl₂ (15 ml) and H₂O (30 ml) were added to the flask. The organic phase was separated. CH₂Cl₂ (30 ml) was added to the aqueous phase, the pH value of the aqueous phase was adjusted to 9-10 with saturated Na₂CO₃ aq. The organic phase was separated, the aqueous phase was re-extracted with CH₂Cl₂ (30 mL×2). The different organic phases were gathered, washed with brine (10 mL), dried with Na₂SO₄, filtered and concentrated under vacuum below 35° C. to afford 0.11 g of crude material 4-amino-1-[(3R,5R)-5-hydroxy-3-piperidyl]-3-(4-phenoxyphenyl)imidazo[4,5-c]pyridin-2-one (Compound of Formula VIb, also referred to above as (11B)) as a foam solid which was not further purified and engaged in Step 12B directly.

Step 12B: To a solution of 4-amino-1-[(3R,5R)-5-hydroxy-3-piperidyl]-3-(4-phenoxyphenyl)imidazo[4,5-c]pyridin-2-one (11B) (0.11 g, 0.26 mmol, 1 eq) in CH₂Cl₂ (44 mL) was added N,N-diisopropylethylamine (177 L, 1.04 mmol, 4.0 eq). The solution was cooled to −5˜0° C., and a solution of trimethyl silyl chloride (33 μL, 0.26 mmol, 1.0 eq) in CH₂Cl₂ (2.5 ml) was added dropwise. The solution was stirred 30 minutes at −5˜0° C., cooled to −25˜−20° C. Acryloyl chloride (27.5 μL, 0.34 mmol, 1.3 eq) in CH₂Cl₂ (2.5 mL) was added dropwise. The reaction was stirred for 10 minutes, warmed to 0˜5° C., and quenched with water (20 mL). The organic phase was separated, washed with brine (10 mL) and concentrated under vacuum below 35° C. to get the crude material. The crude product was purified by prep-TLC (CH₂Cl₂/MeOH=15/1) to get 4-amino-1-[(3R,5R)-5-hydroxy-1-prop-2-enoyl-3-piperidyl]-3-(4-phenoxyphenyl)imidazo[4,5-c]pyridin-2-one (Compound of Formula Ib, also referred to above as (14)) (63 mg, Yield=51%, 99.1% purity) as an off-white powder solid. LC/HRMS: 472.1821 (M+H)+, 470.1790 (M−H)⁻. 1H NMR spectrum (500 MHz, DMSO D6, δ in ppm): 7.76 (d, 1H); 7.50-7.42 (m, 4H); 7.22 (t, 1H), 7.18-7.11 (m, 4H); 6.90 (d, 1H); 6.79 (dd, 1H); 6.13 (dd, 1H); 5.68 (dd, 1H); 5.05 (br. d, 0.75H); 4.96 (br. d, 0.25H); 4.81 (br. s, 2K); 4.65 (br. m, 0.25H); 4.59-4.46 (m, 1.75H); 4.19 (br. d, 0.25H); 4.07 (br. s, 1H); 3.97 (br. d, 0.75H); 3.72 (t, 0.25H); 3.38 (d, 0.75H); 3.32 (m, 0.75H); 2.90 (d, 0.25H); 2.53 (m, 1H); 1.92 (br. d, 1H).

### Example 2. Synthesis of 4-amino-3-(4-phenoxyphenyl)-1-[(3R)-1-(2,3-dihydroxypropanoyl)-3-piperidyl]imidazo[4,5-c]pyridin-2-one

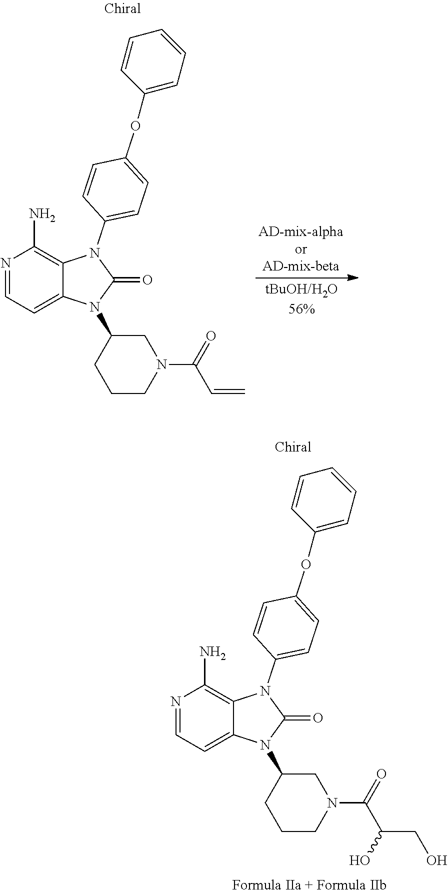 [SMILES: C=CC(=O)N1CCC[C@@H](n2c(=O)n(-c3ccc(Oc4ccccc4)cc3)c3c(N)nccc32)C1 ; Nc1nccc2c1n(-c1ccc(Oc3ccccc3)cc1)c(=O)n2[C@@H]1CCCN(C(=O)C(O)CO)C1]

As shown in the scheme above, Compounds of Formula IIa and IIb were prepared from 4-amino-3-(4-phenoxyphenyl)-1-[(3R)-1-prop-2-enoyl-3-piperidyl]imidazo[4,5-c]pyridin-2-one using AD-Mix-alpha (i.e., (DHQ)2PHAL (hydroquinine 1,4-phthalazinediyl diether, Sigma Aldrich Cat. No. 392723) 0.0016 mole; potassium carbonate, powder 0.4988 mole; potassium ferricyanide 0.4988 mole; potassium osmate dihydrate 0.0007 mol) or AD-Mix-beta (i.e., (DHQD)2PHAL (Sigma Aldrich Cat. No. 392731) 0.0016 mole; potassium carbonate, powder 0.4988 mole; potassium ferricyanide 0.4988 mole; potassium osmate dihydrate 0.0007 mole).

The starting material, 4-amino-3-(4-phenoxyphenyl)-1-[(3R)-1-prop-2-enoyl-3-piperidyl]imidazo[4,5-c]pyridin-2-one, was prepared according to the procedure described in U.S. Pat. No. 9,688,676 at Example 3.

To make the Compounds of Formula IIa and IIb, a suspension of AD-Mix-alpha (154 mg) or AD-Mix-beta (154 mg) in water/tBuOH (1/1, 1 mL/1 mL) was stirred for about 10 minutes until dissolution of the solids. To the yellow solution, under an argon atmosphere, was added the starting material, 4-amino-3-(4-phenoxyphenyl)-1-[(3R)-1-prop-2-enoyl-3-piperidyl]imidazo[4,5-c]pyridin-2-one. Because the starting material was insoluble in the mixture, a yellow suspension was obtained. This suspension was stirred for 20 hours at room temperature. The advancement of the reaction was checked by TLC (AcOEt/MeOH 10/1). The reaction mixture was hydrolyzed with Na₂SO₃, stirred for 20 minutes, extracted with CH₂Cl₂. The organic phase was separated, dried over MgSO₄, filtered and concentrated under vacuum to afford the crude product. The crude product was purified by SCX (strong cation exchange) column (MeOH, then NH₃ 3.5N solution in MeOH) to provide a transparent oil 4-amino-3-(4-phenoxyphenyl)-1-[(3R)-1-(2,3-dihydroxypropanoyl)-3-piperidyl]imidazo[4,5-c]pyridin-2-one as a mixture of two diastereoisomers which were separated by chiral chromatography to provide the Compound of Formula IIa (98% purity) and the Compound of Formula IIb (98% purity). IIa: 1H NMR (500 MHz, DMSO-d6) (2 rotamers 50/50) 6 ppm 1.45-1.65 (m, 1H), 1.80-1.95 (m, 2H), 2.45 (m, 1H), 3.10 (m, 0.5H), 3.25 (m, 0.5H), 3.40-3.55 (m, 2H), 3.67 (m, 0.5H), 4.05-4.50 (m, 4.5H), 4.62 (t, J=4 Hz, 0.5H), 4.77 (t, J=4 Hz, 0.5H), 4.80 (m, 2H), 4.95 (d, J=4 Hz, 0.5H), 5.01 (d, J=4 Hz, 0.5H), 6.90-6.96 (m, 1H), 7.15 (m, 4H), 7.22 (t, J=7 Hz, 1H), 7.45 (m, 4H), 7.75 (d, J=5 Hz, 1H).

IIb: 1H NMR (500 MHz, DMSO-d6) (2 rotamers 50/50) 6 ppm 1.45-1.65 (m, 1H), 1.80-1.95 (m, 2H), 2.45 (m, 1H), 3.10 (m, 0.5H), 3.25 (m, 0.5H), 3.40-3.55 (m, 2H), 3.67 (m, 0.5H), 4.05-4.50 (m, 4.5H), 4.65 (t, J=4 Hz, 0.5H), 4.76 (t, J=4 Hz, 0.5H), 4.80 (m, 2H), 4.98 (m, 1H), 6.90-6.96 (m, 1H), 7.15 (m, 4H), 7.22 (t, J=7 Hz, 1H), 7.45 (m, 4H), 7.75 (d, J=5 Hz, 1H) LC/MS: 490 (M+H)+.

### Example 3. Synthesis of (R)-5-acrylamido-4-(4-amino-2-oxo-3-(4-phenoxyphenyl)-2,3-dihydro-1H-imidazo[4,5-c]pyridin-1-yl)pentanoic acid

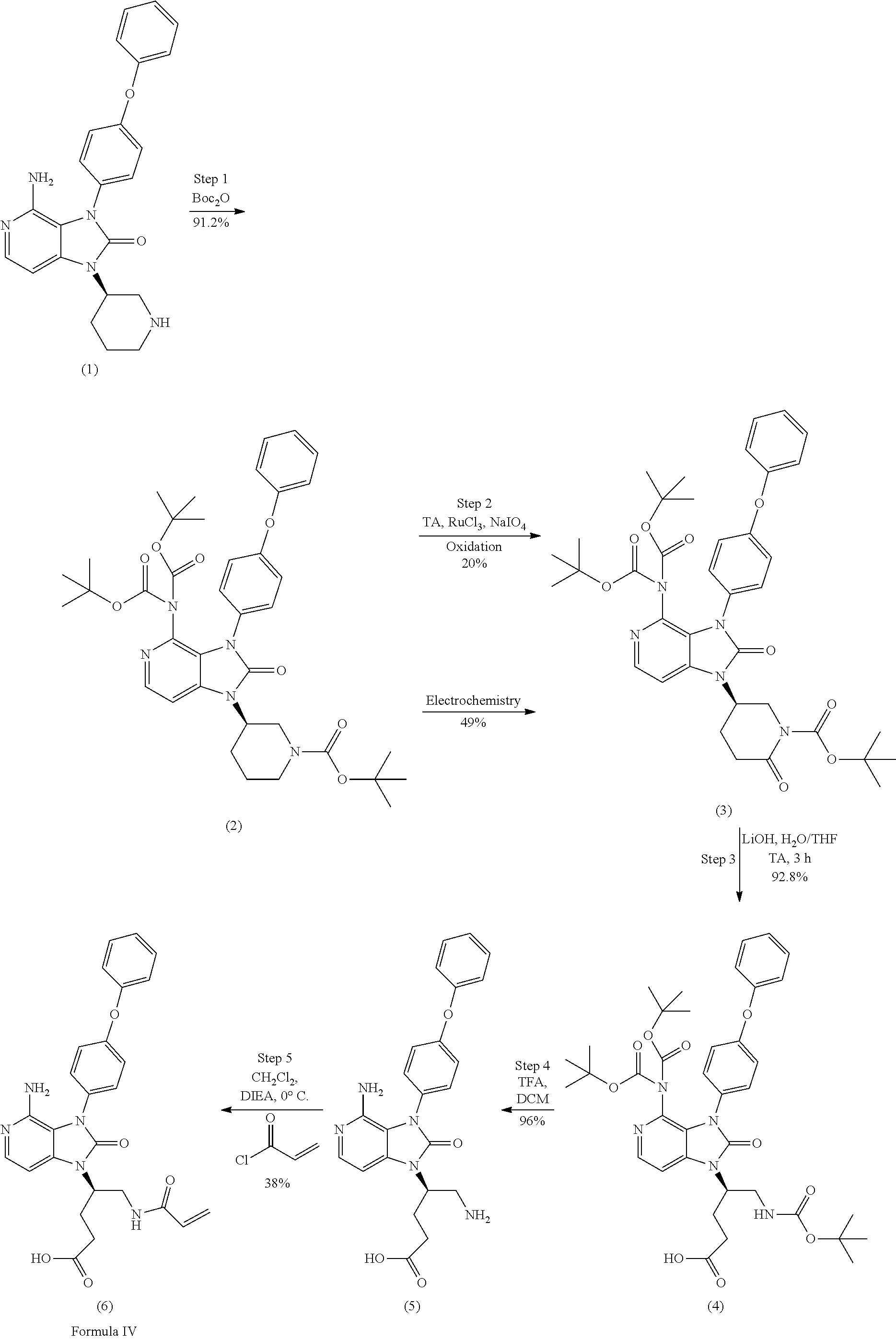

The Compound of Formula IV (referred to above as compound (6)) was prepared as shown in the scheme above and as described in more detail below. The starting material, 4-amino-3-(4-phenoxyphenyl)-1-[(3R)-3-piperidyl]imidazo[4,5-c]pyridin-2-one (1), was prepared according to the procedure described in U.S. Pat. No. 9,688,676 at Example 2. 4-amino-3-(4-phenoxyphenyl)-1-[(3R)-3-piperidyl]imidazo[4,5-c]pyridin-2-one (1) was then converted to an oxalate salt by adding DCM to Compound (1). The solution was switched to EtOH and the resulting solution was reacted with oxalic acid. After filtration, the crude solid was slurried in MTBE, filtered, and dried to afford the oxalate salt of Compound (1).

Step 1: To a flask under an argon atmosphere were added CH₂Cl₂ and the oxalate salt of 4-amino-3-(4-phenoxyphenyl)-1-[(3R)-3-piperidyl]imidazo[4,5-c]pyridin-2-one (1.3 g, 2.66 mmol, 1 eq). To the suspension was then charged DMAP (487 mg, 3.98 mmol, 1.5 eq), DIEA (1.35 mL, 7.97 mmol, 3 eq) and Boc₂O (2.90 g, 13.28 mmol, 5 eq). The starting material dissolved, and the reaction mass became limpid. The solution was stirred at room temperature for 20 hours. The reaction mass was hydrolyzed with water, extracted with CH₂Cl₂. The organic phase was separated, dried over MgSO₄, filtered and concentrated under vacuum to afford the crude product. The crude product was purified by flash chromatography (SiO₂, Hep/AcOEt (1/1)) to give tert-butyl (3R)-3-[4-[bis(tert-butoxycarbonyl)amino]-2-oxo-3-(4-phenoxyphenyl)imidazo[4,5-c]pyridin-1-yl]piperidine-1-carboxylate (2) (1.86 g, yield=91.2%).

Step 2: To a solution of tert-butyl (3R)-3-[4-[bis(tert-butoxycarbonyl)amino]-2-oxo-3-(4-phenoxyphenyl)imidazo[4,5-c]pyridin-1-yl]piperidine-1-carboxylate (2) (1 g, 1.42 mmol, 1 eq) in MeCN/H₂O/CCl₄ (15 mL/20 mL/15 mL) were added NaIO₄ (1.22 g, 5.7 mmol, 4 eq) and RuCl₃·xH₂O (32 mg, 142 mol, 0.1 eq) in that order. The reaction was a light brown mixture. After stirring for 1 hour at room temperature, some more NaIO₄ (152 mg, 0.7 mmol, 0.25 eq) was added. The reaction mass was stirred 24 hours at room temperature, it was dark brown. Upon hydrolyzing with aqueous Na₂S₂O₅ and stirring for 30 minutes, the solution became light yellow. It was extracted with AcOEt. The organic phase was separated, dried over MgSO₄, filtered and concentrated under vacuum to provide the crude product. Purification of the crude product by flash chromatography (SiO₂, Hep/AcOEt (1/1)) provided tert-butyl (3R)-3-[4-[bis(tert-butoxycarbonyl)amino]-2-oxo-3-(4-phenoxyphenyl)imidazo[4,5-c]pyridin-1-yl]piperidine-1-carboxylate (starting material) (450 mg, yield=45%) and tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate (desired product) (3) (200 mg, yield=20%).

Step 2: Electrochemical process—Using Electrasyn 2.0 (IKA) with 20 mL vial setup, to a solution of tert-butyl (3R)-3-[4-[bis(tert-butoxycarbonyl)amino]-2-oxo-3-(4-phenoxyphenyl)imidazo[4,5-c]pyridin-1-yl]piperidine-1-carboxylate (2) (300 mg, 0.43 mmol, 1 eq) in MeCN (12 mL) were added quinuclidine as a mediator (2 eq, 0.86 mmol, 95 mg), 1,1,1,3,3,3-hexafluoropropan-2-ol (“HFIP”) (11 eq, 4.70 mmol, 790 mg) and tetramethylammonium tetrafluoroborate as an electrolyte (2.05 eq, 0.88 mmol, 141 mg). Vial cap set: (Working Electrode) RVC—Reticulated Vitreous Carbon Foam as the anode and Ni Foam as the cathode. The electrochemical reaction was conducted in galvanostatic mode; constant current of 8 mA with no polarity switch. Total load 16 Faradays per mol at room temperature. The reaction was monitored by LC/MS. The reaction was poured into ice water, then a 1M solution of sodium thiosulfate was added. The aqueous phase was washed with ethyl acetate. The organic phase was separated, dried over sodium sulfate, filtered and concentrated under vacuum to provide the crude product. Purification by flash chromatography on silica gel, elution mixture ethyl acetate/pentane (70/30), provided the expected product—tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate (3) (150 mg, yield=49%).

Step 3: To a solution of tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate (3) (820 mg, 1.15 mmol, 1 eq) in THF/H₂O (30 mL/30 mL) was added LiOH (471 mg, 19.4 mmol, 17 eq). The solution was stirred 3 hours at room temperature. The reaction mixture was hydrolyzed with HCl aq 1N until pH=1-2, and then extracted with AcOEt. The organic phase was separated, dried over MgSO₄, filtered and concentrated under vacuum to afford the desired compound: (4R)-4-[4-[bis(tert-butoxycarbonyl)amino]-2-oxo-3-(4-phenoxyphenyl)imidazo[4,5-c]pyridin-1-yl]-5-(tert-butoxycarbonylamino)pentanoic acid (4) (780 mg, Yield=92.8%).

Step 4: In a flask under argon atmosphere, were charged CH₂Cl₂ (5 mL), (4R)-4-[4-[bis(tert-butoxycarbonyl)amino]-2-oxo-3-(4-phenoxyphenyl)imidazo[4,5-c]pyridin-1-yl]-5-(tert-butoxycarbonylamino)pentanoic acid (720 mg, 0.98 mmol, 1 eq) (4), and TFA (2 mL). The mixture was stirred 6 hours at room temperature. After evaporation of the CH₂Cl₂ and the TFA and drying under high vacuum for 10 hours, (4R)-5-amino-4-[4-amino-2-oxo-3-(4-phenoxyphenyl)imidazo[4,5-c]pyridin-1-yl]pentanoic acid (5) (410 mg, 96%) was obtained. It was not further purified and was engaged in next step as is.

Step 5: To a suspension of (4R)-5-amino-4-[4-amino-2-oxo-3-(4-phenoxyphenyl)imidazo[4,5-c]pyridin-1-yl]pentanoic acid (5) (425 mg, 0.98 mmol, 1 eq) and CH₂Cl₂ (6 mL), DMF (0.5 mL) was added in order to solubilize the starting material. Then, the reaction solution was cooled to 0° C., DIEA (343 μL, 1.96 mmol, 2 eq) was charged and a solution of acryloyl chloride (41 μL, 0.49 mmol, 0.5 eq) in CH₂Cl₂ (2 mL) was added dropwise. The solution was allowed to warm to room temperature and stirred for 2 hours. The reaction mass was concentrated under vacuum and directly purified by preparative HPLC, then by supercritical HPLC, to obtain (4R)-4-[4-amino-2-oxo-3-(4-phenoxyphenyl)imidazo[4,5-c]pyridin-1-yl]-5-(prop-2-enoylamino)pentanoic acid (Compound of Formula IV, also referred to above as (6)) (180 mg, Yield=38%, 98% purity). 1H NMR (500 MHz, DMSO-d6) δ ppm 2.02 (m, 1H), 2.17 (m, 2H), 2.28 (m, 1H), 3.65 (m, 2H), 4.36 (m, 1H), 4.78 (s, 2H), 5.53 (dd, J=8, 2 Hz, 1H), 6.02 (dd, J=17, 2 Hz, 1H), 6.09 (dd, J=17, 8 Hz, 1H), 6.65 (m, 1H), 7.15 (d, J=8 Hz, 4H), 7.21 (t, J=7 Hz, 1H), 7.45 (m, 4H), 7.71 (d, J=5 Hz, 1H), 8.28 (t, J=6 Hz, 1H) LC/MS: 488.2 (M+H)+/486.1 (M−H)⁻.

### Example 4. Synthesis of (R)-N-(2-(4-amino-2-oxo-3-(4-phenoxyphenyl)-2,3-dihydro-1H-imidazo[4,5-c]pyridin-1-yl)-5-hydroxypentyl)acrylamide

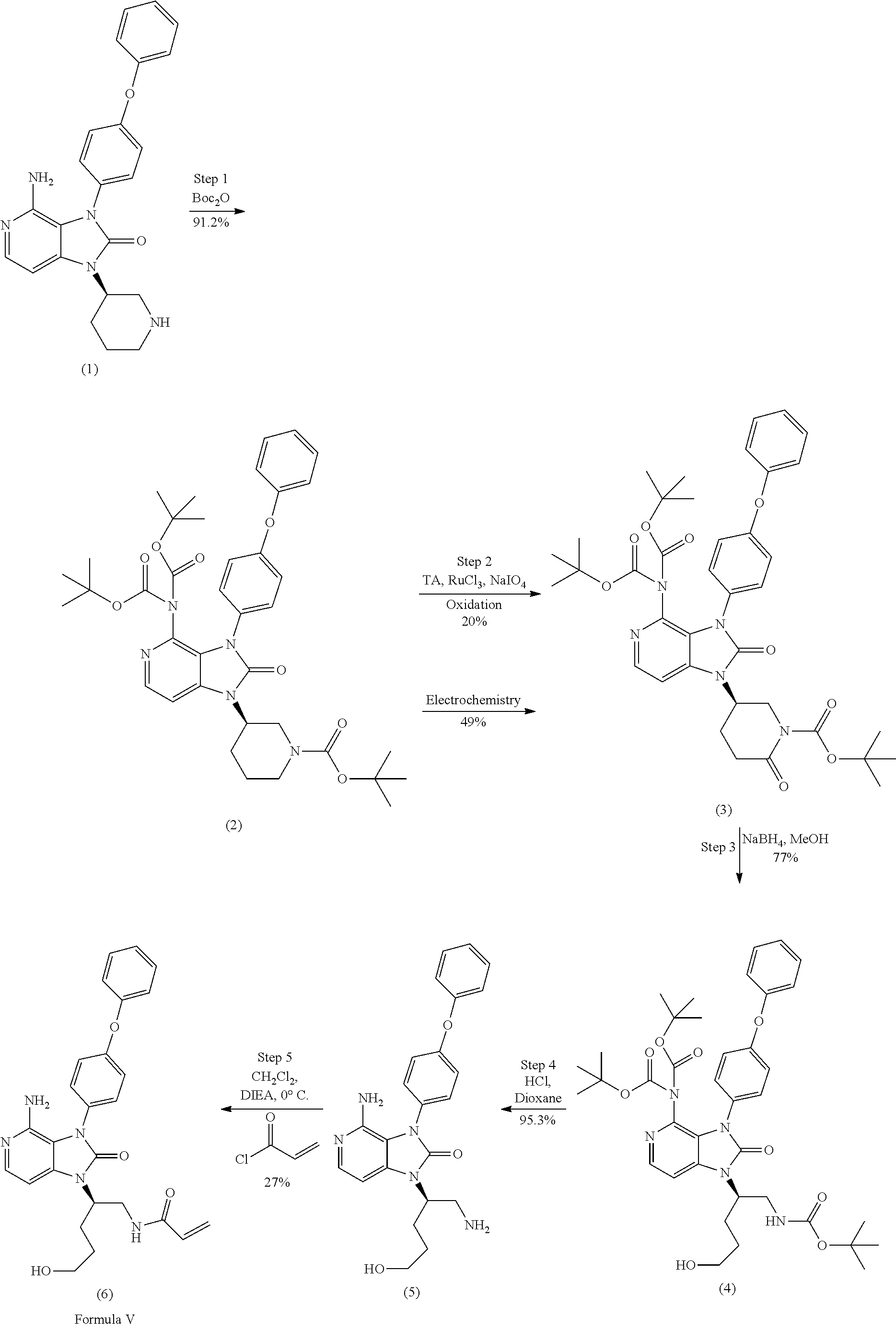

The Compound of Formula V (referred to above as compound (6)) was prepared as shown in the scheme above and as discussed in more detail below.

Steps 1 and 2 were conducted as described in steps 1 and 2 of Example 3. Step 3: To a flask under argon atmosphere at room temperature, were added tert-butyl (5R)-5-[4-[bis(tert-butoxycarbonyl)amino]-2-oxo-3-(4-phenoxyphenyl)imidazo[4,5-c]pyridin-1-yl]-2-oxo-piperidine-1-carboxylate (3) (280 mg, 0.39 mmol, 1 eq), MeOH (8 mL), NaBH₄ (148 mg, 3.9 mmol, 10 eq). The reaction mixture was stirred at room temperature for 3 hours, and then hydrolyzed with H₂O. The organic phase was separated, dried over MgSO₄, filtered and concentrated under vacuum to afford the desired compound tert-butyl (3R)-3-[4-[bis(tert-butoxycarbonyl)amino]-2-oxo-3-(4-phenoxyphenyl)imidazo[4,5-c]pyridin-1-yl]piperidine-1-carboxylate (4) (281 mg, Yield=77%). It was not further purified and directly engaged in the next step.

Step 4: To the starting material tert-butyl N-tert-butoxycarbonyl-N-[1-[(1R)-1-[(tert-butoxycarbonylamino)methyl]-4-hydroxy-butyl]-2-oxo-3-(4-phenoxyphenyl)imidazo[4,5-c]pyridin-4-yl]carbamate (4) (216 mg, 0.30 mmol, 1 eq) was added HCl in dioxane (15 mL). The solution was stirred 2 hours at room temperature. The solvent was evaporated and the crude product 4-amino-1-[(1R)-1-(aminomethyl)-4-hydroxy-butyl]-3-(4-phenoxyphenyl)imidazo[4,5-c]pyridin-2-one (5) (126 mg, Yield=95.3%) was engaged in the next step.

Step 5: To a flask under argon atmosphere were added 4-amino-1-[(1R)-1-(aminomethyl)-4-hydroxy-butyl]-3-(4-phenoxyphenyl)imidazo[4,5-c]pyridin-2-one (125 mg, 0.29 mmol, 1 eq) (5) and CH₂Cl₂ (2 mL). Then, the reaction solution was cooled to 0° C., after charging DIEA (104 μL, 0.59 mmol, 2 eq), and a solution of acryloyl chloride (12.5 μL, 0.15 mmol, 0.5 eq) in CH₂Cl₂ (1 mL) was added dropwise. The solution was kept at 0° C. and stirred for 1 hour. The reaction mass was hydrolyzed with H₂O and extracted with AcOEt. The organic phase was separated, dried over MgSO₄, filtered and concentrated under vacuum to afford the crude compound which was purified by preparative HPLC to give N-[(2R)-2-[4-amino-2-oxo-3-(4-phenoxyphenyl)imidazo[4,5-c]pyridin-1-yl]-5-hydroxy-pentyl]prop-2-enamide (Compound of Formula V, as referred to above as (6)) (38 mg, Yield=27%, >95% purity). 1H NMR (500 MHz, DMSO-d6) δ ppm 1.25-1.40 (m, 2H), 1.83 (m, 1H), 2.08 (m, 1H), 3.35 (m, 2H), 3.50-3.70 (m, 2H), 4.35 (m, 1H), 4.43 (m, 1H), 4.80 (s, 2H), 5.53 (dd, J=8, 2 Hz, 1H), 6.03 (dd, J=17, 2 Hz, 1H), 6.11 (dd, J=17, 8 Hz, 1H), 6.70 (m, 1H), 7.15 (d, J=8 Hz, 4H), 7.22 (t, J=7 Hz, 1H), 7.43 (d, J=7 Hz, 2H), 7.48 (t, J=7 Hz, 2H), 7.74 (d, J=5 Hz, 1H), 8.29 (t, J=6 Hz, 1H) LC/MS: 474.1 (M+H)+.

### Example 5. BTK(h) Study

Compounds of Formula Ib, Formula II, and Formula III were tested against BTK(h) as described herein. All compounds tested were prepared to a working stock of 50× final assay concentration in 100% DMSO. Where appropriate, more concentrated stocks were diluted manually to 50× using 100% DMSO. Compounds, which were powders, were reconstituted to a 10 mM stock in 100% DMSO before further dilution to 50×.

Assay Procedure:

The required volume of the 50× stock of test compound was added to the assay well before a reaction mix containing the enzyme and substrate was added. The reaction was initiated by the addition of ATP at the selected concentration. There was no pre-incubation of the compound with the enzyme/substrate mix prior to ATP addition.

Data Analysis:

Results are expressed as kinase activity remaining, as a percentage of the DMSO control. This is calculated using the following formula:

$$\frac{Mean of Sample Counts - Mean of Blank Counts}{Mean of Control Counts}$$

For IC₅₀ determinations, data are analyzed using XLFit version 5.3 (ID Business Solutions). Sigmoidal dose-response (variable slope) curves are fit based on the mean result for each test concentration using non-linear regression analysis. Where the top and/or bottom of the curve fall >10% out with 100 and 0, respectively, either or both of these limits may be constrained at 100 and 0, provided that the QC criterion on R² is met.

Results are as shown in Table 2.

TABLE 2

| Compound | IC₅₀ (nM) |
|---|---|
| Formula Ib | 34 |
| Formula II | 279 |
| Formula III | 1375 |

### Example 6. In Vitro Studies

Compounds of Formula Ia, Formula Ib, Formula III, Formula IV, and Formula V were tested in various in vitro assays. Compound binding was calculated as a % inhibition of the binding of a radioactively labeled ligand specific for each target. Compound enzyme inhibition effect was calculated as a % inhibition of control enzyme activity. Certain compounds showed significant activity, which was defined in this instance as an inhibition or stimulation higher than 50%, in certain assays.

BZD Binding Assay:

The general procedure is noted below. See Le Fur, G. et al. (1983), Life Sci., 33: 449-457. Kd is affinity of the radioligand for the receptor. Minor variations to the experimental protocol described below may have occurred during the testing, but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| BZD | Rat | [³H]PK | 0.2 nM | 1.8 nM | PK | 15 min | Scintillation |
| (peripheral) | Heart | 11195 | | | 11195 | RT | counting |
| (antagonist | | | | | (10 μM) | | |
| radioligand) | | | | | | | |

The compound of Formula Ia showed significant activity in this assay, exhibiting 52.2% inhibition.

Dopamine Binding Assay:

The general procedure is noted below. See Pristupa, Z. B. et al. (1994), Mol. Pharmacol., 45: 125-135. Minor variations to the experimental protocol described below may have occurred during the testing, but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| Dopamine | Human | [³H]BTCP | 4 nM | 4.5 nM | BTCP | 120 min | Scintillation |
| transporter (h) | recombinant | 11195 | | | (10 μM) | 4° C. | counting |
| (antagonist | (CHO cells) | | | | | | |
| radioligand) | | | | | | | |

The compound of Formula Ia showed significant activity in this assay, exhibiting 65% inhibition. The compound of Formula III also showed significant activity, exhibiting 96.2% inhibition.

D₁ (b) Binding Assay:

The general procedure is noted below. See Zhou, Q. Y. et al. (1990), Nature, 347: 76-80. Minor variations to the experimental protocol described below may have occurred during the testing but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| D₁ (h) | Human | [³H]SCH | 0.3 nM | 0.2 nM | SCH | 60 min | Scintillation |
| (antagonist | recombinant | 23390 | | | 23390 | RT | counting |
| radioligand) | (CHO cells) | | | | (1 μM) | | |

The compound of Formula III showed significant activity in this assay, exhibiting 80% inhibition.

H₂ (b) Binding Assay: The general procedure is noted below. See Leurs, R. et al. (1994), Brit. J. Pharmacol., 112: 847-854. Minor variations to the experimental protocol described below may have occurred during the testing but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| H₂ (h) | Human | [¹²⁵I]APT | 0.075 nM | 2.9 nM | tiotidine | 120 min | Scintillation |
| (antagonist | recombinant | | | | (100 μM) | RT | counting |
| radioligand) | (CHO cells) | | | | | | |

The compound of Formula III showed significant activity in this assay, exhibiting 56.7% inhibition.

δ (DOP) (h) Binding Assay:

The general procedure is noted below. See Simonin, F. et al. (1994), Mol. Pharmacol., 46: 1015-1021. Minor variations to the experimental protocol described below may have occurred during the testing but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| δ (DOP) (h) | Human | [³H] | 0.5 nM | 0.6 nM | naltrexone | 60 min | Scintillation |
| (agonist | recombinant | DADLE | | | (10 μM) | RT | counting |
| radioligand) | (Chem-1 | | | | | | |
| | (RBL) cells) | | | | | | |

The compound of Formula III showed significant activity in this assay, exhibiting 56.7% inhibition.

kappa (h) (KOP) Binding Assay:

The general procedure is noted below. See Simonin, F. et al. (1995), Proc. Natl. Acad. Sci. U.S.A., 92: 7006-7010. Minor variations to the experimental protocol described below may have occurred during the testing but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| kappa (h) | Human | [³H] | 0.5 nM | 0.6 nM | naloxone | 60 min | Scintillation |
| (KOP) | recombinant | U69593 | | | (10 μM) | RT | counting |
| (agonist | (RBL cells) | | | | | | |
| radioligand) | | | | | | | |

The compound of Formula III showed significant activity in this assay, exhibiting 54.6% inhibition.

5-HT₅ₐ (h) Binding Assay:

The general procedure is noted below. See Rees, S. et al. (1994), FEBS Lett., 355: 242-246. Minor variations to the experimental protocol described below may have occurred during the testing but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| 5-HT₅ₐ (h) | Human | [³H] | 1.5 nM | 1.5 nM | serotonin | 120 min | Scintillation |
| (agonist | recombinant | LSD | | | (100 μM) | 37° C. | counting |
| radioligand) | (HEK-293 | | | | | | |
| | cells) | | | | | | |

The compound of Formula III showed significant activity in this assay, exhibiting 56.1% inhibition.

Norepinephrine Transporter (h) Binding Assay:

The general procedure is noted below. See Pacholczyk, T. et al. (1991), Nature, 350: 350-354. Minor variations to the experimental protocol described below may have occurred during the testing but if so, they had no impact on the quality of the results obtained.

| | | | | | Non | | Detection |
|---|---|---|---|---|---|---|---|
| Receptor | Source | Ligand | Conc. | Kd | specific | Incubation | Method |
| Nor- | Human | [³H] | 1 nM | 2.9 nM | desipramine | 120 min | Scintillation |
| epinephrine | recombinant | nisoxetine | | | (1 μM) | 4° C. | counting |
| transporter (h) | (CHO cells) | | | | | | |
| (antagonist | | | | | | | |
| radioligand) | | | | | | | |

The compound of Formula III showed significant activity in this assay, exhibiting 66.7% inhibition.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant FIGURE.

What is claimed is:

1. The compound of Formula Ia: 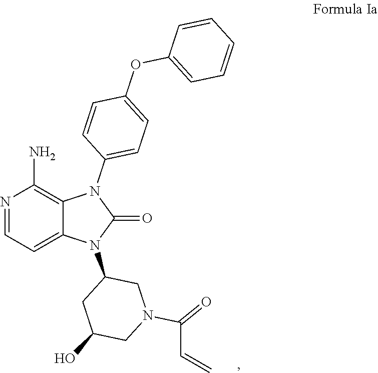 [SMILES: C=CC(=O)N1C[C@@H](O)C[C@@H](n2c(=O)n(-c3ccc(Oc4ccccc4)cc3)c3c(N)nccc32)C1]
or a salt thereof.

2. The compound of claim 1, wherein the compound or salt thereof is about 90% pure.

3. The compound of claim 2, wherein the compound or salt thereof is about 95% pure.

4. The compound of claim 3, wherein the compound or salt thereof is about 98% pure.

5. The compound of Formula Ib: 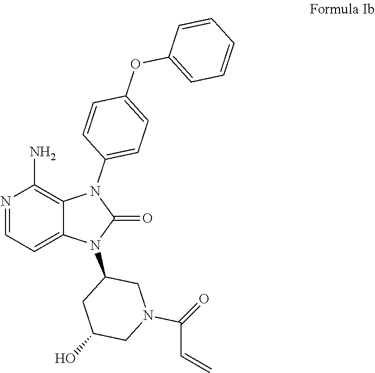 [SMILES: C=CC(=O)N1C[C@H](O)C[C@@H](n2c(=O)n(-c3ccc(Oc4ccccc4)cc3)c3c(N)nccc32)C1]
or a salt thereof, wherein the compound or salt thereof is about 90% pure.

6. The compound of claim 5, wherein the compound or salt thereof is about 95% pure.

7. The compound of claim 6, wherein the compound or salt thereof is about 98% pure.

8. The compound of claim 7, the compound or salt thereof is about 99% pure.

9. A compound of Formula II 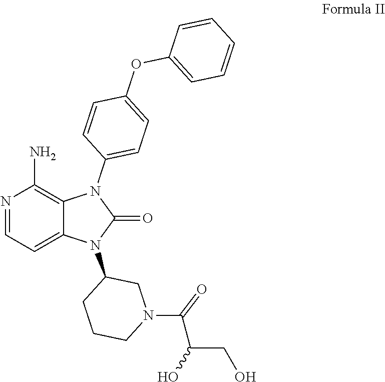 [SMILES: Nc1nccc2c1n(-c1ccc(Oc3ccccc3)cc1)c(=O)n2[C@@H]1CCCN(C(=O)C(O)CO)C1]
or salt thereof, wherein the compound or salt thereof is about 99.6% pure.

10. The compound of claim 9, wherein the compound is 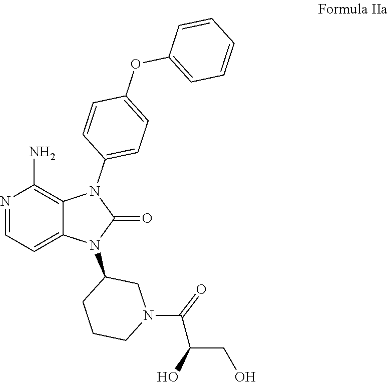 [SMILES: Nc1nccc2c1n(-c1ccc(Oc3ccccc3)cc1)c(=O)n2[C@@H]1CCCN(C(=O)[C@H](O)CO)C1]
or salt thereof, wherein the compound or salt thereof is about 90% pure.

11. The compound of claim 10, wherein the compound of Formula IIa or salt thereof is about 95% pure.

12. The compound of claim 11, wherein the compound of Formula IIa or salt thereof is about 98% pure.

13. The compound of claim 9, wherein the compound is 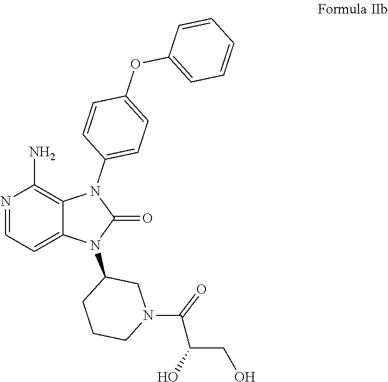 [SMILES: Nc1nccc2c1n(-c1ccc(Oc3ccccc3)cc1)c(=O)n2[C@@H]1CCCN(C(=O)[C@@H](O)CO)C1]
or salt thereof, wherein the compound or salt thereof is about 90% pure.

14. The compound of claim 13, wherein the compound of Formula IIb or salt thereof is about 95% pure.

15. The compound of claim 14, wherein the compound of Formula IIb or salt thereof is about 98% pure.

16. A composition comprising a pharmaceutically acceptable excipient and at least one compound chosen from: 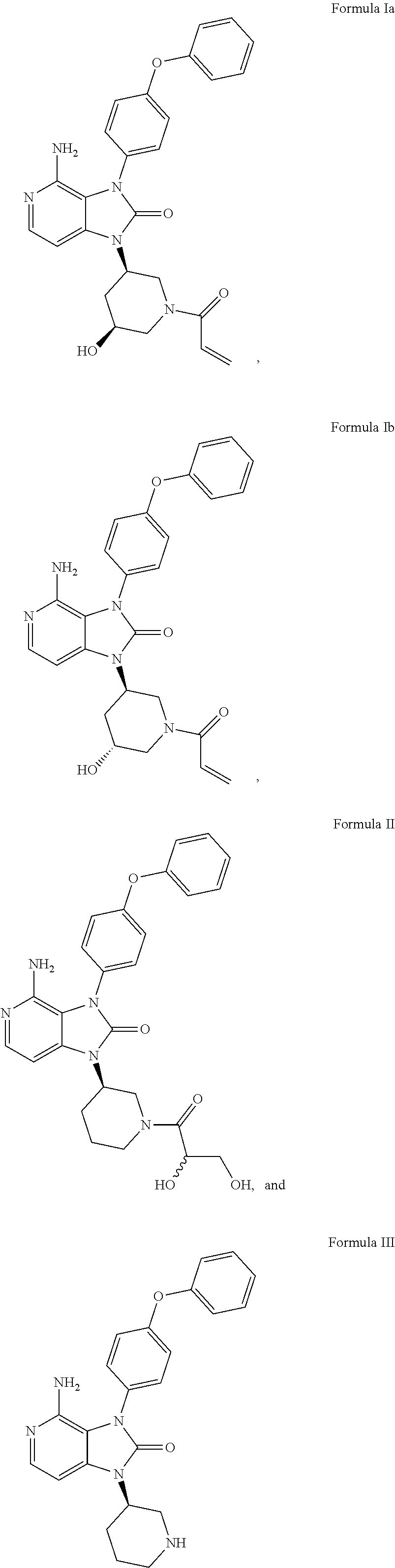 [SMILES: C=CC(=O)N1C[C@@H](O)C[C@@H](n2c(=O)n(-c3ccc(Oc4ccccc4)cc3)c3c(N)nccc32)C1 ; C=CC(=O)N1C[C@H](O)C[C@@H](n2c(=O)n(-c3ccc(Oc4ccccc4)cc3)c3c(N)nccc32)C1 ; Nc1nccc2c1n(-c1ccc(Oc3ccccc3)cc1)c(=O)n2[C@@H]1CCCN(C(=O)C(O)CO)C1 ; Nc1nccc2c1n(-c1ccc(Oc3ccccc3)cc1)c(=O)n2[C@@H]1CCCNC1]
or a salt thereof.

17. The composition of claim 16, wherein at least one compound or salt thereof is present in an effective amount.

18. The composition of claim 16, wherein at least one compound is a compound of Formula Ia or a salt thereof.

19. The composition of claim 18, wherein the compound of Formula Ia or salt thereof is at least about 98% pure.

20. The composition of claim 16, wherein at least one compound is a compound of Formula Ib or a salt thereof.

21. The composition of claim 20, wherein the compound of Formula Ib or salt thereof is at least about 99% pure.

22. The composition of any one of claims 16-21, wherein the composition is in the form of a capsule, tablet, or pill.

23. A method of preparing a compound of Formula Ia or salt thereof, 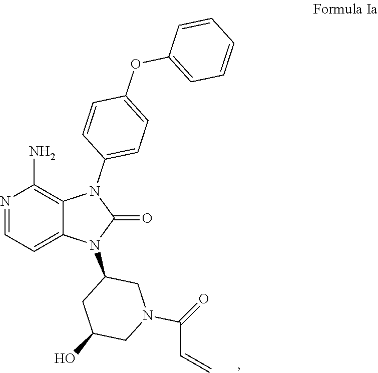 [SMILES: C=CC(=O)N1C[C@@H](O)C[C@@H](n2c(=O)n(-c3ccc(Oc4ccccc4)cc3)c3c(N)nccc32)C1]
the method comprising reacting a compound of Formula VIa 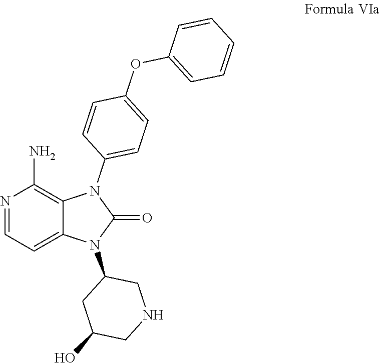 [SMILES: Nc1nccc2c1n(-c1ccc(Oc3ccccc3)cc1)c(=O)n2[C@H]1CNC[C@@H](O)C1]
with acryloyl chloride.

24. The method of claim 23, wherein the method further comprises preparing a compound of Formula VIa from a compound of Formula IX 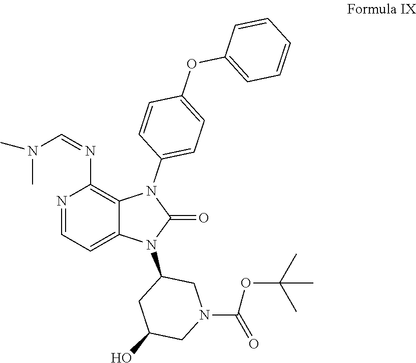 [SMILES: CN(C)/C=N\c1nccc2c1n(-c1ccc(Oc3ccccc3)cc1)c(=O)n2[C@@H]1C[C@H](O)CN(C(=O)OC(C)(C)C)C1]
by acid-based deprotection.

25. A method of preparing a compound of Formula Ib or salt thereof, 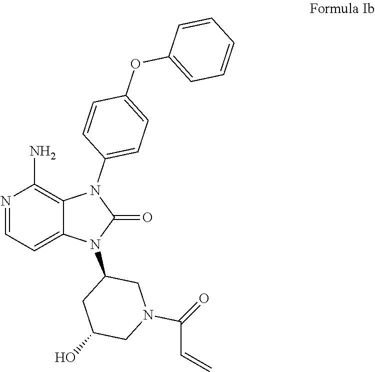 [SMILES: C=CC(=O)N1C[C@H](O)C[C@@H](n2c(=O)n(-c3ccc(Oc4ccccc4)cc3)c3c(N)nccc32)C1]
the method comprising reacting a compound of Formula VIb 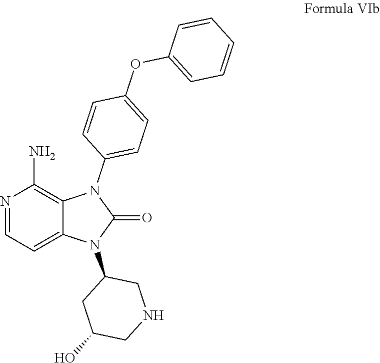 [SMILES: Nc1nccc2c1n(-c1ccc(Oc3ccccc3)cc1)c(=O)n2[C@H]1CNC[C@H](O)C1]
with acryloyl chloride.

26. The method of claim 25, wherein the method further comprises preparing a compound of Formula VIb from a compound of Formula VII 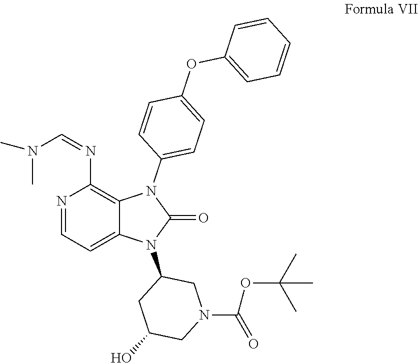 [SMILES: CN(C)/C=N\c1nccc2c1n(-c1ccc(Oc3ccccc3)cc1)c(=O)n2[C@@H]1C[C@@H](O)CN(C(=O)OC(C)(C)C)C1]
by acid-based deprotection.

27. The method of claim 26, wherein the method further comprises preparing a compound of Formula VII from a compound of Formula VIII 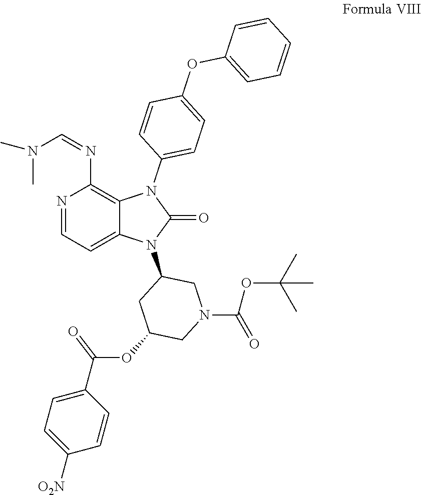 [SMILES: CN(C)/C=N\c1nccc2c1n(-c1ccc(Oc3ccccc3)cc1)c(=O)n2[C@@H]1C[C@@H](OC(=O)c2ccc([N+](=O)[O-])cc2)CN(C(=O)OC(C)(C)C)C1]
by deprotection using a base.

28. The method of claim 27, wherein the method further comprises preparing a compound of Formula VIII from a compound of Formula IX 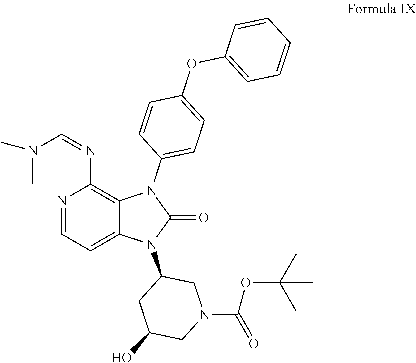 [SMILES: CN(C)/C=N\c1nccc2c1n(-c1ccc(Oc3ccccc3)cc1)c(=O)n2[C@@H]1C[C@H](O)CN(C(=O)OC(C)(C)C)C1]
with triphenylphosphine and an azodicarboxylate.

29. The method of claim 28, wherein the azodicarboxylate is chosen from diethyl azodicarboxylate and diisopropyl azodicarboxylate.

30. The method of claim 29, wherein the azodicarboxylate is diisopropyl azodicarboxylate.

31. A method of treating an autoimmune disorder comprising administering to a patient in need thereof an effective amount of a compound chosen from a compound of Formula Ia, Formula Ib, Formula II, and Formula III 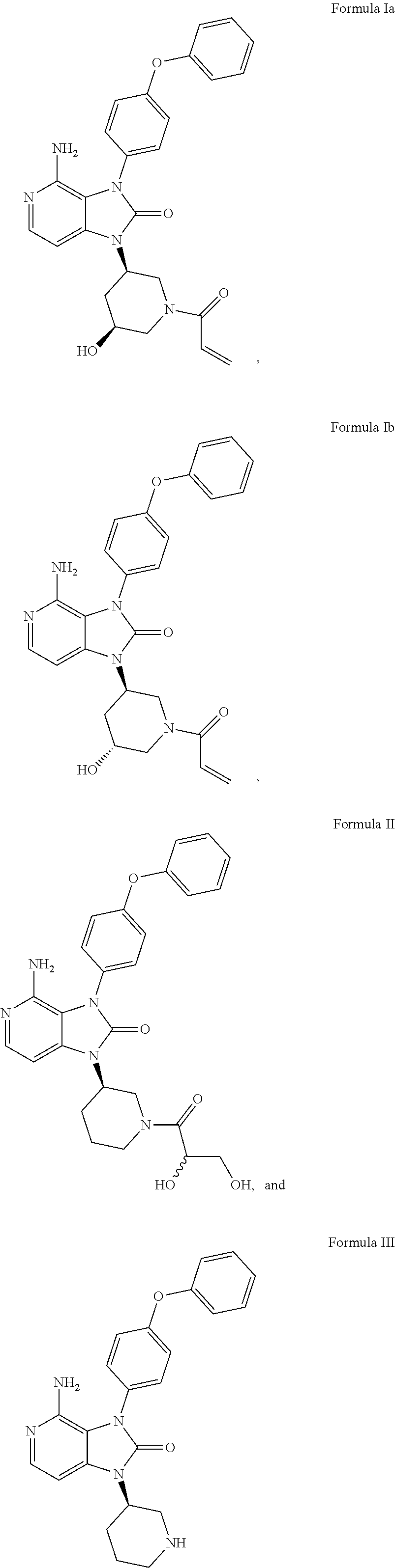 [SMILES: C=CC(=O)N1C[C@@H](O)C[C@@H](n2c(=O)n(-c3ccc(Oc4ccccc4)cc3)c3c(N)nccc32)C1 ; C=CC(=O)N1C[C@H](O)C[C@@H](n2c(=O)n(-c3ccc(Oc4ccccc4)cc3)c3c(N)nccc32)C1 ; Nc1nccc2c1n(-c1ccc(Oc3ccccc3)cc1)c(=O)n2[C@@H]1CCCN(C(=O)C(O)CO)C1 ; Nc1nccc2c1n(-c1ccc(Oc3ccccc3)cc1)c(=O)n2[C@@H]1CCCNC1]
or a salt thereof.

32. The method of claim 31, wherein the autoimmune disorder is selected from the group consisting of lupus, pemphigus vulgaris, myasthenia gravis, Sjogren's syndrome, dry eye, multiple sclerosis, Wegener's granulomatosis, autoimmune hemolytic anemia, idiopathic thrombocytopenic purpura, Granulomatosis with Polyangiitis, and rheumatoid arthritis.

33. The method of claim 31, wherein the autoimmune disorder is lupus or rheumatoid arthritis.

34. A composition for use as a medicament for treating a patient in need thereof comprising an effective amount of at least one compound chosen from 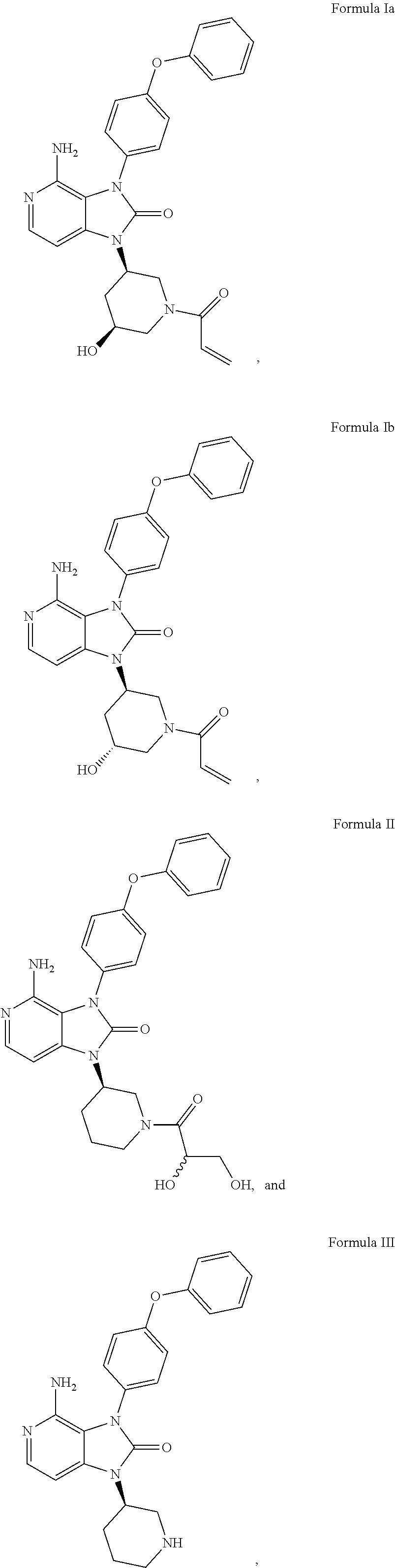 [SMILES: C=CC(=O)N1C[C@@H](O)C[C@@H](n2c(=O)n(-c3ccc(Oc4ccccc4)cc3)c3c(N)nccc32)C1 ; C=CC(=O)N1C[C@H](O)C[C@@H](n2c(=O)n(-c3ccc(Oc4ccccc4)cc3)c3c(N)nccc32)C1 ; Nc1nccc2c1n(-c1ccc(Oc3ccccc3)cc1)c(=O)n2[C@@H]1CCCN(C(=O)C(O)CO)C1 ; Nc1nccc2c1n(-c1ccc(Oc3ccccc3)cc1)c(=O)n2[C@@H]1CCCNC1]
or salt thereof, for treating autoimmune disorders.

35. The composition of claim 34, wherein the autoimmune disorder is selected from the group consisting of lupus, pemphigus vulgaris, myasthenia gravis, Sjogren's syndrome, dry eye, multiple sclerosis, Wegener's granulomatosis, autoimmune hemolytic anemia, idiopathic thrombocytopenic purpura, Granulomatosis with Polyangiitis, and rheumatoid arthritis.

36. The composition of claim 34, wherein the autoimmune disorder is lupus or rheumatoid arthritis.
